(19) United States
(12) Patent Application Publication
(10) Pub. No.: US 2011/0092529 A1
(43) Pub. Date: Apr. 21, 2011

(54) 1,2,4-Triazole Derivatives and Their Use as Oxytocin Antagonists

(75) Inventors: Alan Daniel Brown, Sandwich (GB); Andrew Antony Calabrese, Kent (GB); David Ellis, Kent (GB)

(73) Assignee: Pfizer Inc.

(21) Appl. No.: 11/817,755

(22) PCT Filed: Jan. 6, 2006
(86) PCT No.: PCT/IB2006/600520

§ 371 Date: Sep. 20, 2007

Related U.S. Application Data
(60) Provisional application No. 60/662,651, filed on Mar. 16, 2005.

(30) Foreign Application Priority Data
Mar. 4, 2005 (GB) 0504556.2

(51) Int. Cl. A61K 31/438; A61P 15/00; C07D 401/14; C07D 471/10; A61K 31/4747; C07D 471/20; C07D 513/20; C07D 491/107

(52) U.S. Cl. 514/278; 546/15; 546/17; 546/18; 544/6

(57) ABSTRACT

The present invention relates to a class of substituted triazoles of formula (I) with activity as oxytocin antagonists, uses thereof, processes for the preparation thereof and compositions containing said inhibitors. These inhibitors have utility in a variety of therapeutic areas including sexual dysfunction, particularly premature ejaculation (P.E.).

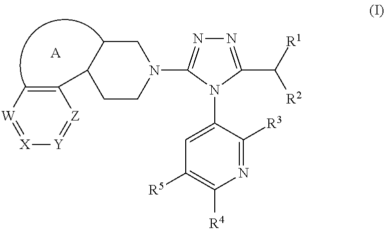

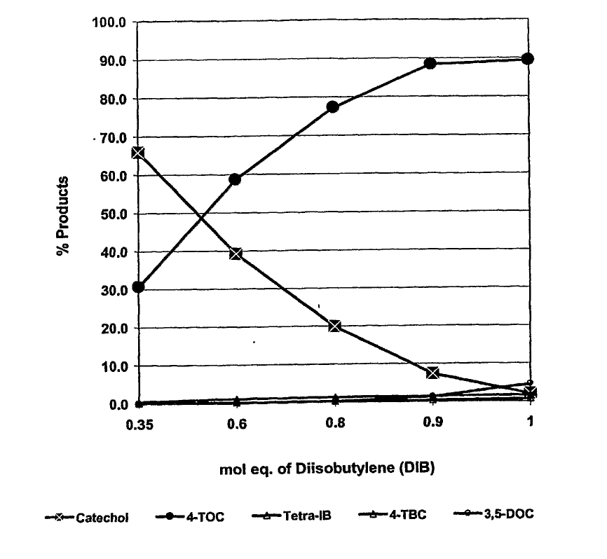

The present invention relates to a class of substituted 1,2,4-triazoles with activity as oxytocin antagonists, uses thereof, processes for the preparation thereof and compositions containing said inhibitors. These inhibitors have utility in a variety of therapeutic areas including sexual dysfunction, particularly premature ejaculation (P.E.).

The present invention provides for compounds of formula (I)

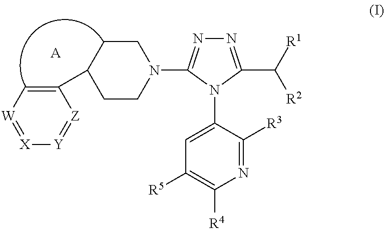

wherein
ring A represents a 5-7 membered carbocyclic or heterocyclic ring containing 1-3 heteroatoms selected from N, O and S; said rings being optionally substituted with one or more groups independently selected from oxo, halo, (C₁-C₆)alkyl, (C₁-C₆)alkoxy, (C₁-C₆)alkoxy(C₁-C₆)alkyl, cyano, NR⁷R⁶, and C(O)NR⁷R⁸;
W, X, Y and Z, which may be the same or different, represent C—R⁶ or N;
R¹ is selected from
(i) H;
(ii) (C₁-C₆)alkyl, which is optionally substituted by O(C₁-C₆)alkyl or phenyl;
(iii) O(C₁-C₆)alkyl, which is optionally substituted by O(C₁-C₆)alkyl;
(iv) NH(C₁-C₆)alkyl, said alkyl group being optionally substituted by O(C₁-C₆)alkyl;
(v) N((C₁-C₆)alkyl)₂, wherein one or both of said alkyl groups may be optionally substituted by O(C₁-C₆)alkyl;
(vi) a 5-8 membered N-linked saturated or partially saturated heterocycle containing 1-3 heteroatoms, each independently selected from N, O and S, wherein at least one heteroatom is N and said ring may optionally incorporate one or two carbonyl groups; said ring being optionally substituted with one or more groups selected from CN, halo, (C₁-C₆)alkyl, O(C₁-C₆)alkyl, C(O)(C₁-C₆)alkyl, C(O)OR⁷, NR⁷R⁸ and C(O)NR⁷R⁸; and
(vii) a 5-7 membered N-linked aromatic heterocycle containing 1-3 heteroatoms each independently selected from N, O and S, wherein at least one heteroatom is N; said ring being optionally substituted with one or more groups selected from CN, halo, (C₁-C₆)alkyl, O(C₁-C₆)alkyl, C(O)(C₁-C₆)alkyl, C(O)OR⁷, NR⁷R⁸ and C(O)NR⁷R⁸;
   R² is selected from H, (C₁-C₆)alkyl and (C₁-C₆)alkoxy(C₁-C₆)alkyl;
   R³, R⁴, R⁵ and R⁶ are each independently selected from H, halo, (C₁-C₆)alkyl, (C₁-C₆)alkoxy, (C₁-C₆)alkoxy(C₁-C₆)alkyl, CN, NR⁷R⁶, and C(O)NR⁷R⁸; and
   R⁷ and R⁸, which may be the same or different, are H or (C₁-C₆)alkyl;
   a tautomer thereof or a pharmaceutically acceptable salt, solvate or polymorph of said compound or tautomer.

Unless otherwise indicated, alkyl and alkoxy groups may be straight or branched and contain 1 to 6 carbon atoms and preferably 1 to 4 carbon atoms. Examples of alkyl include methyl, ethyl, n-propyl, isopropyl, n-butyl, isobutyl, sec-butyl, pentyl and hexyl. Examples of alkoxy include methoxy, ethoxy, isopropoxy and n-butoxy.

Halo means fluoro, chloro, bromo or iodo and is preferably fluoro.

A heterocycle may be saturated, partially saturated or aromatic. Examples of heterocyclic groups are tetrahydrofuranyl, thiolanyl, pyrrolidinyl, pyrrolinyl, imidazolidinyl, imidazolinyl, sulfolanyl, dioxolanyl, dihydropyranyl, tetrahydropyranyl, piperidinyl, pyrazolinyl, pyrazolidinyl, dioxanyl, morpholinyl, dithianyl, thiomorpholinyl, piperazinyl, azepinyl, oxazepinyl, thiazepinyl, thiazolinyl and diazapanyl. Examples of aromatic heterocyclic groups are pyrrolyl, furanyl, thiophenyl, pyrazolyl, imidazolyl, isoxazolyl, oxazolyl, isothiazolyl, thiazolyl, 1,2,3-triazolyl, 1,2,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl and triazinyl. Examples of bicyclic aromatic heterocyclic groups are benzofuranyl, benzothiophenyl, indolyl, benzimidazolyl, indazolyl, benzotriazolyl, quinolinyl and isoquinolinyl.

Unless otherwise indicated, the term substituted means substituted by one or more defined groups. In the case where groups may be selected from a number of alternative groups, the selected groups may be the same or different.

Preferred aspects of the invention are defined below.

In a preferred aspect, the present invention comprises compounds of formula (I) which has the formula (Ia):

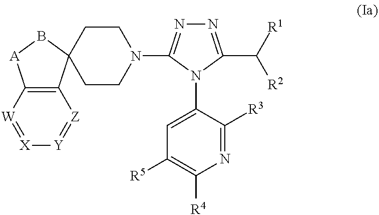

wherein -A-B- is selected from:
—(CH₂)ₘ—, —O(CH₂)ₙ—, —(CH₂)ₙO—, —CH₂OCH₂—, —C(O)O(CH₂)ₚ, —CH₂C(O)O—, —NH(CH₂)ₙ—, —(CH₂)ₙNH—, —CH₂NHCH₂—, —C(O)NH(CH₂)ₚ, —CH₂C(O)NH—, —(CH₂)ₚNHC(O)—, —NHC(O)CH₂—, —S(O)₂NH(CH₂)ₚ, —CH₂S(O)₂NH—, —(CH₂)ₚNHS(O)₂— and —NHS(O)₂CH₂—;
m=2-4; n=1-3; p=0-1;
each CH₂ is optionally substituted by a group independently selected from (C₁-C₆)alkyl, (C₁-C₆)alkoxy, (C₁-C₆)alkoxy(C₁-C₆)alkyl, cyano, NR⁷R⁸, and C(O)NR⁷R⁸;
each NH is optionally substituted by (C₁-C₆)alkyl or (C₁-C₆)alkoxy(C₁-C₆)alkyl; and
W, X, Y, Z, R¹, R², R³, R⁴, R⁵, R⁶, R⁷ and R⁸ are as hereinbefore defined;
a tautomer thereof or a pharmaceutically acceptable salt, solvate or polymorph of said compound or tautomer.

In another preferred aspect, the present invention comprises compounds of formula (Ia):

wherein -A-B- is selected from:
—(CH₂)ₘ—, —O(CH₂)ₙ—, —(CH₂)ₙO—, —CH₂OCH₂—, —NH(CH₂)ₙ—, —(CH₂)ₙNH—, —CH₂NHCH₂—, —C(O)NH(CH₂)ₚ, —CH₂C(O)NH—, —(CH₂)ₚNHC(O)—, —NHC(O)CH₂—, —S(O)₂NH(CH₂)ₚ, —CH₂S(O)₂NH—, —(CH₂)ₚNHS(O)₂— and —NHS(O)₂CH₂—;
m=2-4; n=1-3; p=0-1;
each CH₂ is optionally substituted by (C₁-C₆)alkyl or (C₁-C₆)alkoxy; and each NH is optionally substituted by (C₁-C₆)alkyl or (C₁-C₆)alkoxy(C₁-C₆)alkyl;
W, X, Y and Z are each independently selected from CH, C-halo, C—(C₁-C₃)alkyl, C—(C₁-C₃)alkoxy, C—CN and N;
R¹ is selected from:
(i) H;
(ii) (C₁-C₃)alkyl, which is optionally substituted by O(C₁-C₃)alkyl;
(iii) O(C₁-C₃)alkyl, which is optionally substituted by O(C₁-C₃)alkyl;
(iv) NH(C₁-C₃)alkyl, said alkyl group being optionally substituted by O(C₁-C₃)alkyl; and
(v) N((C₁-C₃)alkyl)₂, wherein one or both of said alkyl groups may be optionally substituted by O(C₁-C₃)alkyl;
   R² is selected from H, (C₁-C₆)alkyl and (C₁-C₆)alkoxy(C₁-C₆)alkyl;
   R³, R⁴ and R⁵ are each independently selected from H, halo, (C₁-C₆)alkyl, (C₁-C₆)alkoxy, (C₁-C₆)alkoxy(C₁-C₆)alkyl, CN, NR⁷R⁸, and C(O)NR⁷R⁸; and
   R⁷ and R⁸, which may be the same or different, are H or (C₁-C₆)alkyl;
   a tautomer thereof or a pharmaceutically acceptable salt, solvate or polymorph of said compound or tautomer.

In another preferred aspect, the present invention comprises compounds of formula (Ia):

wherein -A-B- is selected from:
—O(CH₂)ₙ—, —(CH₂)ₙO—, —CH₂OCH₂—, —NH(CH₂)ₙ—, —(CH₂)ₙNH—, —CH₂NHCH₂—, —C(O)NH(CH₂)ₚ, —CH₂C(O)NH—, —(CH₂)ₚNHC(O)—, —NHC(O)CH₂—, —S(O)₂NH— and —NHS(O)₂—;
n=1-2; p=0-1; and each NH is optionally substituted by methyl;
W, X, Y and Z are each independently selected from CH, C—F, C—Cl; C—CH₃, C—OCH₃, C—CN and N;
R¹ is selected from:
(i) H;
(ii) (C₁-C₃)alkyl, which is optionally substituted by O(C₁-C₃)alkyl; and
(iii) O(C₁-C₃)alkyl, which is optionally substituted by O(C₁-C₃)alkyl;
   R² is H or (C₁-C₃)alkyl; and
   R³, R⁴ and R⁵ are each independently selected from H, halo, (C₁-C₃)alkyl and O(C₁-C₃)alkyl;
   a tautomer thereof or a pharmaceutically acceptable salt, solvate or polymorph of said compound or tautomer.

In another preferred aspect, the present invention comprises compounds of formula (Ia):

wherein -A-B- is selected from:
—O(CH₂)ₙ—, —(CH₂)ₙO—, —CH₂OCH₂—, —NH(CH₂)₂—, —(CH₂)₂NH—, —CH₂NHCH₂—, —C(O)NHCH₂—, —CH₂C(O)NH—, —CH₂NHC(O)— and —S(O)₂NH—;
n=1-2; and each NH is optionally substituted by methyl;
W and X are each independently CH or C—F; Y is selected from CH, C—F and C—CH₃; and Z is CH or N;
R¹ is selected from H, CH₃, OCH₃, OCH₂CH₃, OCH(CH₃)₂, OCH₂CH₂OCH₃;

### R² is H or CH₃; and

R³, R⁴ and R⁵ are each independently selected from H, chloro, fluoro, methyl and methoxy;
a tautomer thereof or a pharmaceutically acceptable salt, solvate or polymorph of said compound or tautomer.

Preferred embodiments of the compounds of formula (I) or (Ia) according to the above aspects are those that incorporate one or more of the following preferences.

Preferably, -A-B- is selected from:

—(CH₂)ₘ—, —O(CH₂)ₙ—, —(CH₂)ₙO—, —CH₂OCH₂—, —NH(CH₂)ₙ—, —(CH₂)ₙNH—, —CH₂NHCH₂—, —C(O)NH(CH₂)ₚ, —CH₂C(O)NH—, —(CH₂)ₚNHC(O)—, —NHC(O)CH₂—, —S(O)₂NH(CH₂)ₚ, —CH₂S(O)₂NH—, —(CH₂)ₚNHS(O)₂— and —NHS(O)₂CH₂—;
m=2-4; n=1-3; p=0-1;
each CH₂ is optionally substituted by (C₁-C₆)alkyl or (C₁-C₆)alkoxy; and each NH is optionally substituted by (C₁-C₆)alkyl or (C₁-C₆)alkoxy(C₁-C₆)alkyl.

More preferably, -A-B- is selected from:

—(CH₂)ₘ—, —O(CH₂)ₙ—, —(CH₂)ₙO—, —CH₂OCH₂—, —NH(CH₂)ₙ—, —(CH₂)ₙNH—, —CH₂NHCH₂—, —C(O)NH(CH₂)ₚ, —CH₂C(O)NH—, —(CH₂)ₚNHC(O)—, —NHC(O)CH₂—, —S(O)₂NH(CH₂)ₚ, —CH₂S(O)₂NH—, —(CH₂)ₚNHS(O)₂— and —NHS(O)₂CH₂—;
m=2-4; n=1-3; p=0-1; and each CH₂ or NH is optionally substituted by methyl.

Yet more preferably, -A-B- is selected from:

—O(CH₂)ₙ—, —(CH₂)ₙO—, —CH₂OCH₂—, —NH(CH₂)ₙ—, —(CH₂)ₙNH—, —CH₂NHCH₂—, —C(O)NH(CH₂)ₚ, —CH₂C(O)NH—, —(CH₂)ₚNHC(O)—, —NHC(O)CH₂—, —S(O)₂NH— and —NHS(O)₂—;
n=1-2; p=0-1; and each NH is optionally substituted by methyl.

Even more preferably, -A-B- is selected from:

—O(CH₂)ₙ—, —(CH₂)ₙO—, —CH₂OCH₂—, —NH(CH₂)₂—, —(CH₂)₂NH—, —CH₂NHCH₂—, —C(O)NHCH₂—, —CH₂C(O)NH—, —CH₂NHC(O)— and —S(O)₂NH—;
n=1-2; and each NH is optionally substituted by methyl.

Most preferably, -A-B- is selected from:

—O(CH₂)ₙ—, —(CH₂)ₙO—, —CH₂OCH₂— and —(CH₂)₂NCH₃—; and n=1-2.

Preferably, W, X, Y and Z are each independently selected from CH, C-halo, C—(C₁-C₆)alkyl, C—(C₁-C₆)alkoxy, C—(C₁-C₆)alkoxy(C₁-C₆)alkyl, C—CN and N.

More preferably, W, X, Y and Z are each independently selected from CH, C-halo, C—(C₁-C₃)alkyl, C—(C₁-C₃)alkoxy, C—CN and N.

Yet more preferably, W, X, Y and Z are each independently selected from CH, C—F, C—Cl, C—(C₁-C₃)alkyl, C—(C₁-C₃)alkoxy, C—CN and N.

Even more preferably, W, X, Y and Z are each independently selected from CH, C—F, C—Cl, C—CH₃, C—OCH₃, C—CN and N.

Even more preferably, W, X, Y and Z are each independently selected from CH, C—F, C—CH₃, C—OCH₃ and N.

Most preferably, W and X are each independently CH or C—F; Y is selected from CH, C—F and C—CH₃; and Z is CH or N.

In a preferred aspect, W, X, Y and Z are CH.

In another preferred aspect, W, X and Y are CH and Z is N.

In another preferred aspect, W, X and Z are CH and Y is C—CH₃.

In another preferred aspect, W, Y and Z are CH and X is C—F.

In another preferred aspect, X and Z are CH and W and Y are C—F.

Preferably, R¹ is selected from:
(i) H;
(ii) (C₁-C₃)alkyl, which is optionally substituted by O(C₁-C₃)alkyl;
(iii) O(C₁-C₃)alkyl, which is optionally substituted by O(C₁-C₃)alkyl;
(iv) NH(C₁-C₃)alkyl, said alkyl group being optionally substituted by O(C₁-C₃)alkyl; and
(v) N((C₁-C₃)alkyl)₂, wherein one or both of said alkyl groups may be optionally substituted by O(C₁-C₃)alkyl.

More preferably, R¹ is selected from:
(i) H;
(ii) (C₁-C₃)alkyl, which is optionally substituted by O(C₁-C₃)alkyl; and
(iii) O(C₁-C₃)alkyl, which is optionally substituted by O(C₁-C₃)alkyl.

Yet more preferably, R¹ is selected from H, CH₃, OCH₃, OCH₂CH₃, OCH(CH₃)₂, OCH₂CH₂OCH₃.

Most preferably, R¹ is selected from H, methyl and methoxy.

Preferably, R² is H or (C₁-C₃)alkyl.

More preferably, R² is H or CH₃.

Most Preferably, R² is H.

Preferably, R³, R⁴ and R⁵ are each independently selected from H, halo, (C₁-C₆)alkyl and O(C₁-C₆)alkyl.

More preferably, R³, R⁴ and R⁵ are each independently selected from H, halo, (C₁-C₃)alkyl and O(C₁-C₃)alkyl.

Yet more preferably, R³, R⁴ and R⁵ are each independently selected from H, chloro, fluoro, methyl and methoxy.

Most preferably, R³ and R⁵ are both H and R⁴ is methoxy.

Preferred compounds of formula (I) are:
1′-[5-(methoxymethyl)-4-(6-methoxypyridin-3-yl)-4H-1,2,4-triazol-3-yl]-3H-spiro[2-benzofuran-1,4′-piperidine];
1′-[5-(methoxymethyl)-4-(6-methoxypyridin-3-yl)-4H-1,2,4-triazol-3-yl]spiro[1-benzofuran-3,4′-piperidine];
5-fluoro-1′-[4-(6-methoxypyridin-3-yl)-5-methyl-4H-1,2,4-triazol-3-yl]-3H-spiro[2-benzofuran-1,4′-piperidine];
1′-[4-(6-methoxypyridin-3-yl)-5-methyl-4H-1,2,4-triazol-3-yl]spiro[1-benzofuran-3,4′-piperidine];
1′-[4-(6-methoxypyridin-3-yl)-5-methyl-4H-1,2,4-triazol-3-yl]-3H-spiro[2-benzofuran-1,4′-piperidine]; and
5-fluoro-1′-[5-(methoxymethyl)-4-(6-methoxypyridin-3-yl)-4H-1,2,4-triazol-3-yl]-3H-spiro[2-benzofuran-1,4′-piperidine];
   and tautomers thereof and pharmaceutically acceptable salts, solvates and polymorphs of said compound or tautomer.

Pharmaceutically acceptable salts of the compounds of formula (I) comprise the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Included within the scope of the invention are complexes such as clathrates, drug-host inclusion complexes wherein the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘pro-drugs’. Further information on the use of prodrugs may be found in “Pro-drugs as Novel Delivery Systems”, Vol. 14, ACS Symposium Series (T. Higuchi and W. Stella) and “Bioreversible Carriers in Drug Design”, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Pro-drugs” by H. Bundgaard (Elsevier, 1985).

Some examples of pro-drugs in accordance with the invention include

(i) where the compound of formula I contains a carboxylic acid functionality, an ester thereof, for example, a compound wherein the hydrogen of the carboxylic acid functionality of the compound of formula (I) is replaced by (C₁-C₈)alkyl; and
(ii) where the compound of formula (I) contains a primary or secondary amino functionality, an amide thereof, for example, a compound wherein, as the case may be, one pr both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references. Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃—>—CH₂OH);
(ii) where the compound of formula (I) contains an alkoxy group, an hydroxy derivative thereof (—OR—>—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂—>COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, a keto group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

The present invention includes all crystal forms of the compounds of formula (I) including racemates and racemic mixtures (conglomerates) thereof. Stereoisomeric conglomerates may be separated by conventional techniques known to those skilled in the art—see, for example, “Stereochemistry of Organic Compounds” by E. L. Eliel and S. H. Wilen (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are intermediate compounds as hereinafter defined, all salts, solvates and complexes thereof and all solvates and complexes of salts thereof as defined hereinbefore for compounds of formula (I). The invention includes all polymorphs of the aforementioned species and crystal habits thereof.

When preparing compounds of formula (I) in accordance with the invention, it is open to a person skilled in the art to routinely select the form of intermediate which provides the best combination of features for this purpose. Such features include the melting point, solubility, processability and yield of the intermediate form and the resulting ease with which the product may be purified on isolation.

Compounds of the invention intended for pharmaceutical use may be administered as crystalline or amorphous products or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelabie backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999). Other means of topical administration include delivery by electroporation, iontophoresis, phonophoresis, sonophoresis and microneedle or needle-free (e.g. Powderject™, Bioject™, etc.) injection. Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or, hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 2 to 30 mg of the compound of formula (I). The overall daily dose will typically be in the range 50 to 100 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 50 mg to 100 mg depending, of course, on the mode of administration and efficacy. For example, oral administration may require a total daily dose of from 50 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

### Processes

In the following general methods, R¹, R², R³, R⁴, R⁵, R⁸, ring A, W, X, Y, and Z are as previously defined for a compound of formula (I) unless otherwise stated. Compounds of general formula (I) may be prepared according to reaction Scheme 1.

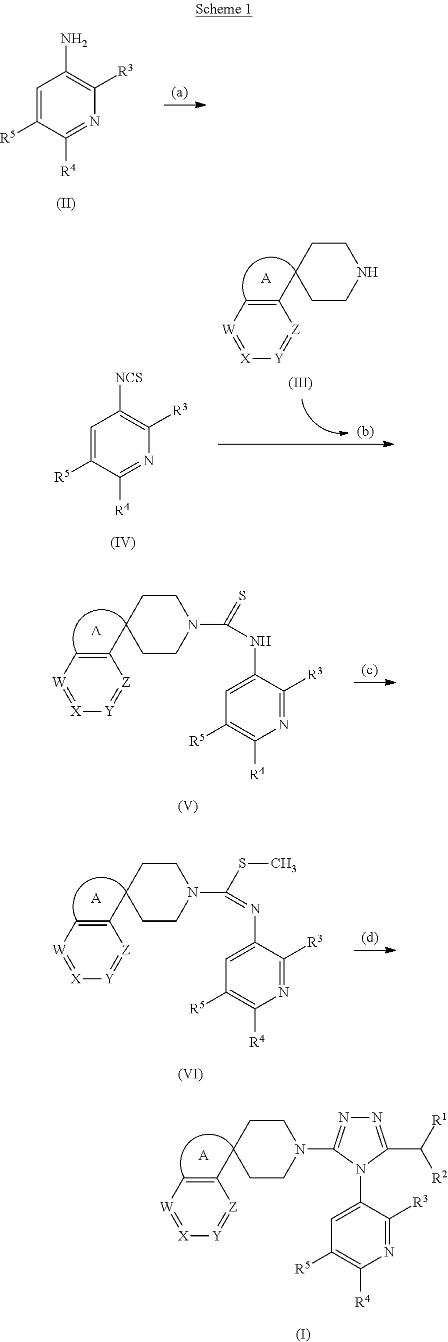

Compounds of formula (II) are available commercially.

### Step (a): Formation of Isothiocyanate:

The amine of formula (II) is treated with an appropriate thiocarbonyl transfer reagent, such as 1,1′-thiocarbonyldi-2(1H)-one or 1,1′-thiocarbonyldiimidazole, to provide the isothiocyanate of formula (IV). The reaction is performed in a suitable solvent, such as dichloromethane, at between 0° C. and room temperature for between 0.5 and 4 hours.

Preferred conditions comprise reacting 1 eq. of (II) with 1 to 1.4 eq. of 1,1′-thiocarbonyldi-2(1H)-one in dichloromethane at between 0° C. and room temperature for between 0.5 and 4 hrs.

### Step (b): Formation of Thiourea:

The isothiocyanate of formula (IV) is treated with an equimolar amount of the amine of formula (III) to provide the thiourea of formula (V). The reaction is performed in a suitable solvent, such as dichloromethane or ethanol, at room temperature for up to 72 hrs.

Preferred conditions comprise reacting 1 eq. of amine (III) with 1 eq. of isothiocyanate (IV) in dichloromethane at room temperature for up to 72 hrs.

Alternatively, the compound of formula (V) may be prepared from the amines of formula (II) and (III) in a one-pot procedure, without isolation of the isothiocyanate of formula (IV).

Preferred conditions comprise reacting 1 eq. of amine (II) with 1 eq. of 1,1′-thiocarbonyldi-2(1H)-one in dichloromethane at between 0° C. and room temperature for up to 3 hrs, followed by reacting with 1 eq. of amine (III) at room temperature for up to 72 hrs.

### Step (c): Alkylation of Thiourea:

Compounds of formula (VI) may be prepared by methylation of the thiourea of formula (V) using a suitable methylating agent, such as methyl iodide or methyl tosylate, in the presence of a suitable base, such as KO^{t}Bu in a suitable solvent, such as THF or ether at between 0° C. and the reflux temperature of the solvent for about 18 hrs.

Preferred conditions comprise reacting 1 eq. of (V), 1 to 1.2 eq. of KO^{t}Bu and 1 to 1.2 eq. of methyl tosylate, in THF at room temperature for up to 4 hrs.

### Step (d): Triazole Formation:

Compounds of formula (I) may be prepared by reaction of compounds of formula (VI) with a suitable acyl hydrazide (R¹R²CHCONHNH₂) optionally under acidic catalysis, such as TFA or p-TSA, in a suitable solvent, such as THF or n-BuOH, at between room temperature and the reflux temperature of the solvent.

Preferred conditions comprise reacting a catalytic amount of TFA with 1 eq. of (VI) and 2 eq. of acyl hydrazide (R¹R²CHCONHNH₂) in THF at between room temperature and the reflux temperature of the solvent for up to 25 hrs.

Compounds of formula (III) may be prepared according to methods known in the literature (e.g. Marzabadi et al. WO 04/004714, or Kubota et al. Chem. Pharm. Bull. 46(2); 242-253 and 351-354; 1998 or Mills et al. U.S. Pat. No. 5,962,462) and references therein. Alternatively, compounds of formula (III), may be prepared as described in Schemes 2 to 5 below.

Compounds of formula (III) where ring A contains an O atom adjacent to the piperidine ring may be prepared by the methods shown in Scheme 2, below.

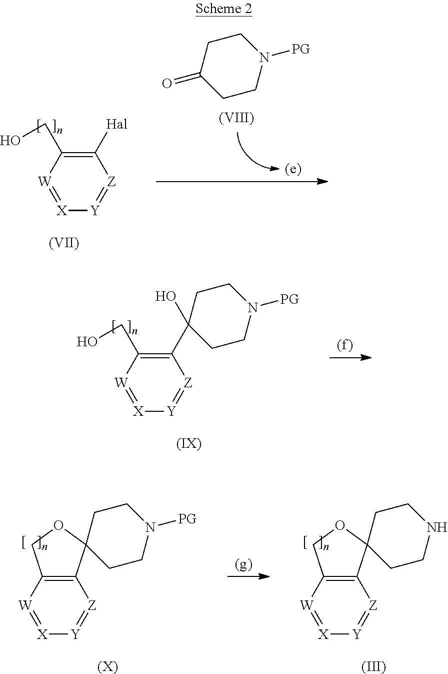

PG represents a suitable nitrogen protecting group, typically BOC or benzyl and preferably BOC and n represents 1, 2 or 3.

Hal represents a halogen atom, typically Cl or Br and preferably Br.

Compounds of formula (VII) are available commercially, or may be prepared by analogy with methods known in the literature. e.g. Ashimori et. al. Chem. Pharm. Bull. 38; 9; 1990; 2446.

Compounds of formula (VIII) are available commercially or may be prepared using standard methodology.

Step (e): Compounds of formula (IX) may be prepared by formation of a suitable dianion of the alcohol of formula (VII) followed by reaction of this dianion with the piperidinone of formula (VIII). Typically, the dianion may be formed by reaction with 2 to 3 equivalents of a suitable strong base, such as n-BuLi, t-BuLi, in a suitable solvent, such as THF, n-heptane or ether, at low temperature for about 3 hrs. This is then treated with the compound of formula (VIII) at between low temperature and room temperature for about 18 hours.

Preferred conditions comprise reacting 2 eq. of n-BuLi with 1 eq. of (VII) in THF or ether at between −70° C. and room temperature for about 3 hrs followed by addition of 1.1 eq. of (VIII) at between −70° C. and room temperature and stirring for about 18 hrs.

### Step (f): Ring Closure:

Compounds of formula (X) may be obtained by dehydration of the compound of formula (IX) under basic or acidic conditions, optionally in the presence of a dehydrating agent, via preparation of an intermediate suitable leaving group, such as mesylate. Typically, the alcohol of formula (IX) is converted to a suitable leaving group, such as methane sulfonyl or tosyl, in the presence of a suitable base, such as triethylamine or Hünig's base, in an appropriate solvent, such as toluene or dichloromethane, at between 0° C. and room temperature for up to 24 hours, which reacts in-situ to provide the compound of formula (X).

Preferred conditions comprise reacting (IX) with 1.1 to 1.6 eq. of methane sulfonyl chloride and 2 eq. of triethylamine in dichloromethane at between 0° C. and room temperature for up to 24 hours.

### Step (g): Removal of Protecting Group:

The compound of formula (III) may be obtained by removal of the nitrogen protecting group using standard methodology, as described in “Protecting Groups in Organic Synthesis” by T. W. Greene and P. Wutz.

When PG represents BOC, preferred conditions comprise reacting compound (X) with an equi-volumetric solution of TFA and dichloromethane at between 0° C. and room temperature for up to 2 hrs.

Alternatively, compounds of formula (III) where ring A contains an oxygen atom adjacent to the aromatic ring may be prepared by the methods shown in Scheme 3 below.

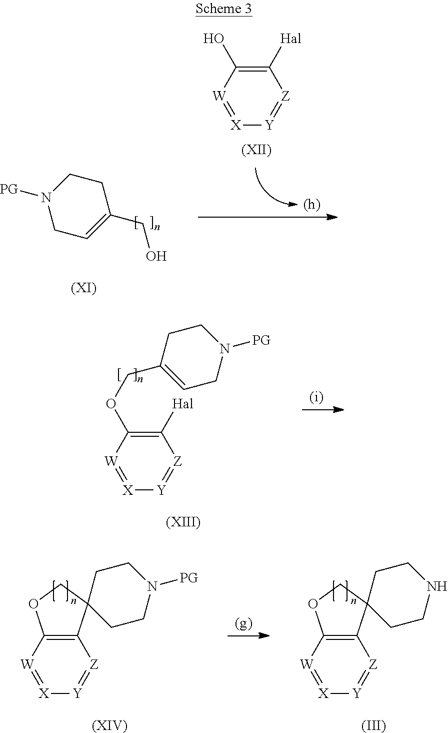

PG represents a suitable nitrogen protecting group, typically BOC or benzyl and preferably benzyl and n represents 1, 2 or 3.

Hal represents a halogen atom, typically Cl or Br and preferably Br.

Compounds of formula (XI) may be obtained using literature procedures, (e.g. by analogy with the methods found in WO 01/87838, WO 94/20459 and WO 01/053258).

### Step (h): Mitsunobu Reaction:

Compounds of formula (XIII) may be prepared by reaction of the compounds of formulae (XI) and (XII) in a Mitsunobu reaction using standard methodology. In a typical procedure the compounds of formulae (XI) and (XII) are treated with a suitable phosphine such as tri-ⁿbutylphosphine or triphenyl phosphine, followed by a suitable dehydrogenating agent, typically an azo compound such as diisopropyl azodicarboxylate (DIAD) or di-tert-butyl azodicarboxylate, in a solvent such as dichloromethane, tetrahydrofuran or N,N-dimethylformamide, at temperatures between 25 and 115° C. for 1 to 48 hours, optionally in the absence of light. Preferred conditions comprise reacting 1 eq. of (XII), 1.05 eq. of (XI), 1.2 eq. of PPh₃ and 1.1 eq. of DIAD, in THF in the absence of light, between 0° C. and room temperature for up to 24 hrs.

### Step (i): C—C Bond Formation:

Compounds of formula (XIV) may be prepared by a radical initiated cyclisation of the compound of formula (XIII), in the presence of a suitable radical initiator, such as azoisobutyronitrile (AIBN), and a radical carrier source, such as Bu₃SnH or (Me₃Si)₃SiH, in a suitable solvent, such as toluene, at elevated temperature for about 4 hours.

Preferred conditions comprise reacting 1 eq. of (XIII), a sub-stoichiometric quantity of AIBN and 4 eq. of Bu₃SnH, in toluene at the reflux temperature of the solvent for about 3 hrs.

### Step (g): Removal of Protecting Group:

The compound of formula (III) may be obtained by removal of the nitrogen protecting group using standard methodology, as described in “Protecting Groups in Organic Synthesis” by T. W. Greene and P. Wutz.

When PG represents benzyl, typically this may be achieved by catalytic hydrogenation in the presence of a suitable catalyst, such as Pd/C, in a suitable alcoholic solvent, such as water, methanol or ethanol, at between room temperature and about 60° C. under an atmosphere of hydrogen. Alternatively, this may be achieved by transfer hydrogenation in the presence of a suitable catalyst, such as Pd/C, and a hydrogen donor, such as formic acid or NH₄CO₂H, in a suitable solvent, such as ethanol or methanol at elevated temperature.

Preferred conditions comprise treating 1 eq. of compound (XIV) with 5 eq. of NH₄CO₂H over 10% Pd/C in ethanol at reflux temperature for about 1.5 hrs, or hydrogenating 1 eq. of compound (XIV) over 10% Pd/C in ethanol:water (9:1) by volume at 60° C. and 60 psi hydrogen.

Alternatively, compounds of formula (III) where ring A contains a nitrogen atom adjacent to the piperidine ring may be prepared by the methods shown in Scheme 4 below.

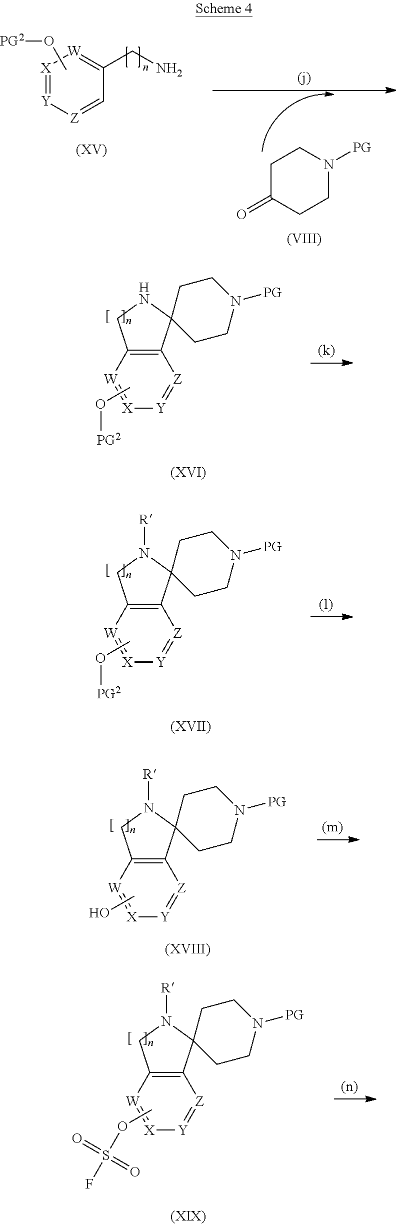

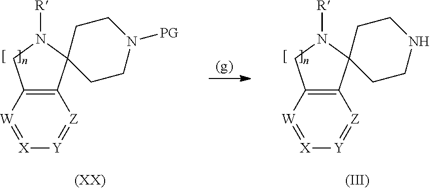

PG represents a suitable nitrogen protecting group, typically BOC or benzyl and preferably benzyl. PG² represents a suitable alcohol protecting group, typically an alkyl group, such as methyl, methoxymethyl or benzyl, and preferably methyl and n represents 1, 2 or 3. Compounds of formula (XV) are available commercially or may be prepared using standard chemical transformations.

### Step (j): Cyclisation Reaction:

Compounds of formula (XVI) may be prepared by reaction of the compounds of formulae (XV) and (VIII), optionally in the presence of acid, such as TFA, HCl or phosphoric acid, and optionally in the presence of solvent, such as ethanol, at elevated temperature for up to 18 hrs. Preferred conditions comprise reacting 1 eq. of (XV) and 1.15 eq. of (VIII) in phosphoric acid under reflux for about 16 hrs.

### Step (k): Reductive Amination Reaction:

Compounds of formula (XVII), wherein R′ is (C₁-C₆)alkyl or (C₁-C₆)alkoxy(C₁-C₆)alkyl, may be prepared by reaction of the appropriate aldehyde/ketone with an amine of formula (XVI) to form an intermediate imine compound, which is reduced by a suitable reducing agent, such as NaCN(BH)₃ or Na(OAc)₃BH, optionally in the presence of NaOAc, optionally in the presence of a drying agent, such as molecular sieves or MgSO₄, in a suitable solvent, such as tetrahydrofuran, methanol or dichloromethane at room temperature for 3 to 72 hrs. Preferred conditions comprise reacting 1 eq. of (XVI) with an excess of the appropriate aldehyde or ketone and 1.5 eq. of Na(OAc)₃BH in methanol for up to 24 hrs.

### Step (l): Removal of Protecting Group:

The compound of formula (XVIII) may be obtained by removal of the hydroxy protecting group using standard methodology, as described in “Protecting Groups in Organic Synthesis” by T. W. Greene and P. Wutz. When PG represents methyl, preferred conditions comprise reacting 1 eq. of (XVI) in excess HBr_{(aq)} in acetic acid at reflux temperature for about 24 hrs.

### Step (m): Formation of Triflate:

Compounds of formula (XIX) may be prepared by treatment of the alcohol of formula (XVIII) with a slight excess of triflating agent, such as triflic anhydride, in the presence of a suitable base, such as triethylamine, 4-methyl morpholine or Hünig's base, in a suitable solvent, such as dichloromethane or ethyl acetate at between 0° C. and room temperature for between 1 and 18 hrs.

Preferred conditions comprise reacting 1 eq. of (XIX) with 1.1 eq. of triflic anhydride and 1.16 eq. Hünig′ s base in dichloromethane at between 0° C. and room temperature for about 4 hrs.

### Step (n): Removal of Triflate:

Compounds of formula (XX) may be prepared by reduction of compounds of formula (XIX) in the presence of a suitable hydride donor, such as triethylsilane, a suitable catalyst, such as Pd(OAc)₂, and a chelating ligand, such as 1,2-bis-(diphenylphosphino)propane (dppp), in a suitable solvent, such as DMF, at elevated temperature. Preferred conditions comprise reacting 1 eq. of (XIX) with 2.5 eq. of triethylsilane, a catalytic amount of Pd(OAc)₂ and a catalytic amount of dppp in DMF at about 60° C. for about 1 hr.

Step (g): Compounds of formula (III) may be obtained from compounds of formula (XX) by analogy with the methods previously described for step (g), Scheme 3.

Alternatively, compounds of formula (III), where ring A contains a nitrogen atom adjacent to the piperidine ring and when R⁶ represents an activating group, such as (C₁-C₆)alkyl, may be prepared by the methods shown in Scheme 5 below.

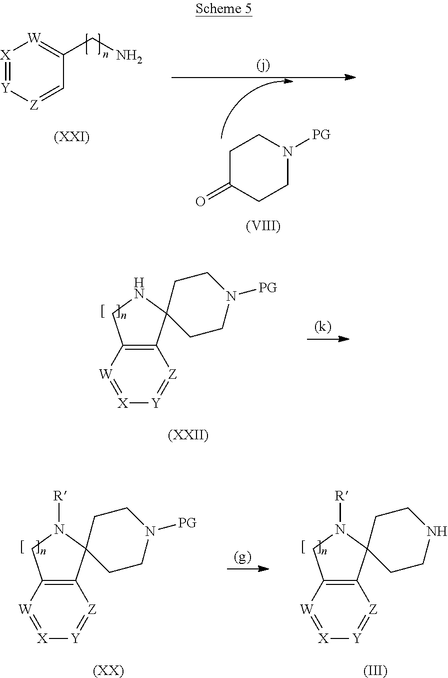

PG represents a suitable nitrogen protecting group, typically BOC or benzyl and preferably benzyl and n represents 1, 2 or 3. Compounds of formula (XXI) are available commercially or may be prepared using standard chemical transformations.

Compounds of formula (XXII) may be prepared from the amine of formula (XXI) and the piperidone of formula (VIII) using the methods previously described in step (j) above. Compounds of formula (XX) may be prepared from compounds of formula (XXII) using the methods previously described in step (k) above. Compounds of formula (III) may be prepared from compounds of formula (XX) using the methods previously described in step (g) above.

All of the above reactions and the preparations of novel starting materials disclosed in the preceding methods are conventional and appropriate reagents and reaction conditions for their performance or preparation as well as procedures for isolating the desired products will be well known to those skilled in the art with reference to literature precedents and the examples and preparations hereto.

### Utility

The compounds of the invention are useful because they have pharmacological activity in mammals, including humans. More particularly, they are useful in the treatment or prevention of a disorder in which modulation of the levels of oxytocin could provide a beneficial effect. Disease states that may be mentioned include sexual dysfunction, particularly premature ejaculation; preterm labour, complications in labour, appetite and feeding disorders, benign prostatic hyperplasia, premature birth, dysmenorrhoea, congestive heart failure, arterial hypertension, liver cirrhosis, nephrotic hypertension, ocular hypertension, obsessive compulsive disorder and neuropsychiatric disorders.

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al, J. Urology, 1999, 161, 5-11).

FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders, Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Thus, in accordance with a further aspect of the invention, there is provided the use of a compound of the invention in the preparation of a medicament for the treatment or prophylaxis of hypoactive sexual desire disorder, sexual arousal disorder, orgasmic disorder and sexual pain disorder, more preferably for the treatment or prophylaxis of sexual arousal disorder, orgasmic disorder, and sexual pain disorder, and most preferably in the treatment or prophylaxis of sexual arousal disorder.

Hypoactive sexual desire disorder is present if a woman has no or little desire to be sexual, and has no or few sexual thoughts or fantasies. This type of FSD can be caused by low, testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

The Diagnostic and Statistical Manual (DSM) IV of the American Psychiatric Association defines Female Sexual Arousal Disorder (FSAD) as being:
“a persistent or recurrent inability to attain or to maintain until completion of the sexual activity adequate lubrication-swelling response of sexual excitement. The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, peri- and post menopausal (±HRT) women. It is associated with concomitant disorders such as depression, cardiovascular diseases, diabetes and UG disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

Male sexual dysfunction (MSD) is generally associated with either erectile dysfunction, also known as male erectile dysfunction (MED) and/or ejaculatory disorders such as premature ejaculation, anorgasmia (unable to achieve orgasm) or desire disorders such as hypoactive sexual desire disorder (lack of interest in sex).

PE is a relatively common sexual dysfunction in men. It has been defined in several different ways but the most widely accepted is the Diagnostic and Statistical Manual of Mental Disorders IV one which states:
“PE is a lifelong persistent or recurrent ejaculation with minimal sexual stimulation before, upon or shortly after penetration and before the patient wishes it. The clinician must take into account factors that affect duration of the excitement phase, such as age, novelty of the sexual partner or stimulation, and frequency of sexual activity. The disturbance causes marked distress of interpersonal difficulty.”

The International Classification of Diseases 10 definition states:
“There is an inability to delay ejaculation sufficiently to enjoy lovemaking, manifest as either of the following: (1) occurrence of ejaculation before or very soon after the beginning of intercourse (if a time limit is required: before or within 15 seconds of the beginning of intercourse); (2) ejaculation occurs in the absence of sufficient erection to make intercourse possible. The problem is not the result of prolonged abstinence from sexual activity”.

Other definitions which have been used include classification on the following criteria:
Related to partner's orgasm
Duration between penetration and ejaculation
Number of thrust and capacity for voluntary control

Psychological factors may be involved in PE, with relationship problems, anxiety, depression, prior sexual failure all playing a role.

Ejaculation is dependent on the sympathetic and parasympathetic nervous systems. Efferent impulses via the sympathetic nervous system to the vas deferens and the epididymis produce smooth muscle contraction, moving sperm into the posterior urethra. Similar contractions of the seminal vesicles, prostatic glands and the bulbouretheral glands increase the volume and fluid content of semen. Expulsion of semen is mediated by efferent impulses originating from a population of lumber spinothalamic cells in the lumbosacral spinal cord (Coolen & Truitt, Science, 2002, 297, 1566) which pass via the parasympathetic nervous system and cause rhythmic contractions of the bulbocavernous, ischiocavemous and pelvic floor muscles. Cortical control of ejaculation is still under debate in humans. In the rat the medial pre-optic area and the paraventricular nucleus of the hypothalamus seem to be involved in ejaculation.

Ejaculation comprises two separate components—emission and ejaculation. Emission is the deposition of seminal fluid and sperm from the distal epididymis, vas deferens, seminal vesicles and prostrate into the prostatic urethra. Subsequent to this deposition is the forcible expulsion of the seminal contents from the urethral meatus. Ejaculation is distinct from orgasm, which is purely a cerebral event. Often the two processes are coincidental.

A pulse of oxytocin in peripheral serum accompanies ejaculation in mammals. In man oxytocin but not vasopressin plasma concentrations are significantly raised at or around ejaculation. Oxytocin does not induce ejaculation itself; this process is 100% under nervous control via α1-adrenoceptor/sympathetic nerves originating from the lumbar region of the spinal cord. The systemic pulse of oxytocin may have a role in the peripheral ejaculatory response. It could serve to modulate the contraction of ducts and glandular lobules throughout the male genital tract, thus influencing the fluid volume of different ejaculate components for example. Oxytocin released centrally into the brain could influence sexual behaviour, subjective appreciation of arousal (orgasm) and latency to subsequent ejaculation.

Accordingly, one aspect of the invention provides for the use of a compound of formula (I), without the proviso, in the preparation of a medicament for the prevention or treatment of sexual dysfunction, preferably male sexual dysfunction, most preferably premature ejaculation.

It has been demonstrated in the scientific literature that the number of oxytocin receptors in the uterus increases during pregnancy, most markedly before the onset of labour (Gimpl & Fahrenholz, 2001, Physiological Reviews, 81 (2), 629-683). Without being bound by any theory it is known that the inhibition of oxytocin can assist in preventing preterm labour and in resolving complications in labour.

Accordingly, another aspect of the invention provides for the use of a compound of formula (I), without the proviso, in the preparation of a medicament for the prevention or treatment of preterm labour and complications in labour.

Oxytocin has a role in feeding; it reduces the desire to eat (Arletti at al., Peptides, 1989, 10, 89). By inhibiting oxytocin it is possible to increase the desire to eat. Accordingly oxytocin inhibitors are useful in treating appetite and feeding disorders.

Accordingly, a further aspect of the invention provides for the use of a compound of formula (I), without the proviso, in the preparation of a medicament for the prevention or treatment of appetite and feeding disorders.

Oxytocin is implicated as one of the causes of benign prostatic hyperplasia (BPH). Analysis of prostate tissue have shown that patients with BPH have increased levels of oxytocin (Nicholson & Jenkin, Adv. Exp. Med. & Biol., 1995, 395, 529). Oxytocin antagonists can help treat this condition.

Accordingly, another aspect of the invention provides for the use of a compound of formula (I), without the proviso, in the preparation of a medicament for the prevention or treatment of benign prostatic hyperplasia.

Oxytocin has a role in the causes of dysmenorrhoea due to its activity as a uterine vasoconstrictor (Akerlund, Ann. NY Acad. Sci., 1994, 734, 47). Oxytocin antagonists can have a therapeutic effect on this condition.

Accordingly, a further aspect of the invention provides for the use of a compound of formula (I), without the proviso, in the preparation of a medicament for the prevention of treatment of dysmenorrhoea.

It is to be appreciated that all references herein to treatment include curative, palliative and prophylactic treatment.

The compounds of the present invention may be coadministered with one or more agents selected from:
1) One or more selective serotonin reuptake inhibitors (SSRIs) such as dapoxetine, paroxetine, 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), N-methyl-N-({3-[3-methyl-4-(methylsulfanyl)phenoxy]-4-pyridinyl}methyl)amine (Example 38, PCT Application no PCT/IB02/01032).
2) One or more local anaesthetics;
3) one or more α-adrenergic receptor antagonists (also known as α-adrenoceptor blockers, α-receptor blockers or α-blockers); suitable α₁-adrenergic receptor antagonists include: phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, Example 19 of WO9830560, terazosin and abanoquil; suitable α₂-adrenergic receptor antagonists include dibenamine, tolazoline, trimazosin; efaroxan, yohimbine, idazoxan clonidine and dibenamine; suitable non-selective α-adrenergic receptor antagonists include dapiprazole; further α-adrenergic receptor antagonists are described in PCT application WO99/30697 published on 14 Jun. 1998 and U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference;
4) one or more cholesterol lowering agents such as statins (e.g. atorvastatin/Lipitor-trade mark) and fibrates;
5) one or more of a serotonin receptor agonist, antagonist or modulator, more particularly agonists, antagonists or modulators for example 5HT1A, 5HT2A, 5HT2C, 5HT3, 5HT6 and/or 5HT7 receptors, including those described in WO-09902159, WO-00002550 and/or WO-00028993;
6) one or more NEP inhibitors, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719; IC50 values against NEP and ACE may be determined using methods described in published patent application EP1097719-A1, paragraphs [0368] to [0376];
7) one or more of an antagonist or modulator for vasopressin receptors, such as relcovaptan (SR 49059), conivaptan, atosiban, VPA-985, CL-385004, Vasotocin.
8) Apomorphine—teachings on the use of apomorphine as a pharmaceutical may be found in U.S. Pat. No. 5,945,117;
9) Dopamine agonists (in particular selective D2, selective D3, selective D4 and selective D2-like agents) such as Pramipexole (Pharmacia Upjohn compound number PNU95666), ropinirole, apomorphine, surmanirole, quinelorane, PNU-142774, bromocriptine, carbergoline, Lisuride;
10) Melanocortin receptor agonists (e.g. Melanotan II and PT141) and selective MC3 and MC4 agonists (e.g. THIQ);
11) Mono amine transport inhibitors, particularly Noradrenaline Re-uptake Inhibitors NRIs) (e.g. Reboxetine), other Serotonin Re-uptake Inhibitors (SRIs) (e.g. paroxetine, dapoxetine) or Dopamine Re-uptake Inhibitors (DRIs);
12) 5-HT_{1A} antagonists (e.g. robalzotan); and
13) PDE inhibitors such as PDE2 (e.g. erythro-9-(2-hydroxyl-3-nonyl)-adenine) and Example 100 of EP 0771799-incorporated herein by reference) and in particular a PDE5 inhibitor such as the pyrazolo[4,3-d]pyrimidin-7-ones disclosed in EP-A-0463756; the pyrazolo[4,3-d]pyrimidin-7-Ones disclosed in EP-A-0526004; the pyrazolo[4,3-d]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453; the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124; the pyrazolo[4,3-d]pyrimidin-7-ones disclosed in published international application WO 01/27112; the pyrazolo[4,3-d]pyrimidin-7-ones disclosed in published international application WO 01/27113; the compounds disclosed in EP-A-1092718 and the compounds disclosed in EP-A-1092719.

Preferred PDE5 inhibitors for use with the invention:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as, 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
5-(2-ethoxy-5-morpholinoacetylphenyl)-1-methyl-3-n-propyl-1,6-dihydro-7H-pyrazolo[4,3-d]pyrimidin-7-one (see EP-A-0526004);
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);
(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperzin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);
5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and
the compound of example 11 of published international application WO93/07124 (EISAI); and
compounds 3 and 14 from Rotella D P, J. Med. Chem., 2000, 43, 1257.

Still further PDE5 inhibitors for use with the invention include:
4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

The contents of the published patent applications and journal articles and in particular the general formulae of the therapeutically active compounds of the claims and exemplified compounds therein are incorporated herein in their entirety by reference thereto.

More preferred PDE5 inhibitors for use with the invention are selected from the group:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); and
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7 one or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof.

A particularly preferred PDE5 inhibitor is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) (also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine) and pharmaceutically acceptable salts thereof. Sildenafil citrate is a preferred salt.

Preferred agents for coadministration with the compounds of the present invention are PDE5 inhibitors, selective serotonin reuptake inhibitors (SSRIs), vasopressin V_{1A} antagonists, α-adrenergic receptor antagonists, NEP inhibitors, dopamine agonists and melanocortin receptor agonists as described above. Particularly preferred agents for coadministration are PDE5 inhibitors, SSRIs, and V_{1A} antagonists as described herein.

### Assay

A suitable assay for determining the oxytocin antagonist activity of a compound is detailed herein below.

### Oxytocin Receptor Beta-Lactamase Assay

### Materials:

### Cell Culture/Reagents

A: cell culture

### Nutrient Mixture

### F12 Ham's

### Foetal Bovine Serum (FBS)

### Geneticin

### Zeocin

### Trypsin/EDTA

PBS (phosphate buffered saline)

### HEPES

B: reagents

### Oxytocin

OT receptor-specific antagonist
Molecular grade Dimethyl Sulphoxide (DMSO)

### Trypan Blue Solution 0.4%

### CCF4-AM (Solution A)

### Pluronic F127s (Solution B)

### 24% PEG, 18% TR40 (Solution C)

### Probenecid (Dissolved at 200 mM in 200 mM NaOH, Solution D)

### Methods

### Cell Culture

Cells used are CHO-OTR/NFAT-β-Lactamase. The NFAT-β-lactamase expression construct was transfected into the CHO-OTR cell line and clonal populations were isolated via fluorescence activated cell sorting (FACS). An appropriate clone was selected to develop the assay.

### Growth Medium

### 90% F12 Nutrient Mix, 15 mM HEPES

### 10% FBS

400 μg/ml Geneticin
200 μg/ml Zeocin

### 2 mM L-Glutamine

### Assay Media

### 99.5% F12 Nutrient Mix, 15 mM HEPES

### 0.5% FBS

Recovery of cells—A vial of frozen cells is thawed rapidly in 37° C. water bath and the cell suspension transferred into a T225 flask with 50 ml of fresh growth medium and then incubated at 37° C., 5% CO₂ in an incubator until the cells adhered to the flask Replace media with 50 ml of fresh growth media the following day.

Culturing cells—CHO—OTR—NFAT-βLactamase cells were grown in growth medium. Cells were harvested when they reached 80-90% confluence removing the medium and washing with pre-warmed PBS. PBS was then removed and Trypsin/EDTA added (3 mls for T225 cm² flask) before incubating for 5 min in 37° C./5% CO₂ incubator. When cells were detached, pre-warmed growth media was added (7 mls for T225 cm² flask) and the cells re-suspended and mixed gently by pipetting to achieve single cell suspension. The cells were split into T225 flask at 1:10 (for 3 days growth) and 1:30 (for 5 days growth) ratio in 35 ml growth medium.

### β-Lactamase Assay Method

### Day 1

### Cell Plate Preparation

Cells grown at 80-90% confluence were harvested and counted. Suspensions of cells at 2×105 cells/ml in growth medium were prepared and 30 μl of cells suspension added in 384-well, black clear-bottom plates. A blank plate containing diluents from each reagent was used for background subtraction.

Plates were incubated at 37° C., 5% CO₂ overnight.

### Day 2

### Cells Stimulation

10 μl antagonist/Compound (diluted in assay media containing 1.25% DMSO=antagonist diluent) was added to appropriate wells and incubated for 15 minutes at 37° C., 5% CO₂.
10 μl oxytocin, made up in assay media, was added to all wells and incubated for 4 hours at 37° C., 5% CO₂.
A separate 384-well cell plate was used to generate an oxytocin dose response curve. (10 μl antagonist diluent was added to every well 10 μl of oxytocin was then added. The cells are then treated as per antagonist/compound cell plates).

Preparation of 1 ml of 6× Loading Buffer with Enhanced Loading Protocol (this requires scale-up according to number of plates to be screened)
12 μl of solution A (1 mM CCF4-AM in Dry DMSO) was added to 60 μl of solution B (100 mg/ml Pluronic-F127 in DMSO+0.1% Acetic Acid) and vortexed.
The resulting solution was added to 925 μl of solution C (24% w/w PEG400, 18% TR40 v/v in water).
75 μl of solution D was added (200 mM probenecid in 200 mM NaOH).
10 μl of 6× Loading Buffer was added to all wells and incubated for 1.5 hrs-2 hrs at room temperature in the dark.
The plates were read using an LJL Analyst, Excitation 405 nm, Emission 450 nm and 530 nm, gain optimal, lagtime 0.40 μs integration, 4 flashes, bottom reading.

Using the assay described above, the compounds of the present invention all exhibit oxytocin antagonist activity, expressed as a Ki value, of less than 1 μM. Preferred examples have Ki values of less than 200 nM and particularly preferred examples have Ki values of less than 50 nM.

The compound of Example 1 has a Ki value of 12.9 nM. The compound of Example 6 has a Ki value of 11.5 nM. The compound of example 17 has a Ki value of 10.2 nM.

The invention is illustrated by the following non-limiting examples in which the following abbreviations and definitions are used:

Arbocel® Filtration agent, from J. Rettenmaier & Sohne, Germany

### BOC Tert-butyloxycarbonyl

### CDCl₃ Chloroform-d1

### d Doublet

dd Doublet of doublets

### DMF Dimethylformamide

### DMSO Dimethylsulfoxide

ES+ Electrospray ionisation positive scan.
eq. Equivalent

### ¹H NMR Proton Nuclear Magnetic Resonance Spectroscopy

### LRMS (Low Resolution) Mass Spectroscopy

### m Multiplet

m/z Mass spectrum peak

### q Quartet

### s Singlet

### t Triplet

TFA Trifluoroacetic acid

### THF Tetrahydrofuran

p-TSA para-toluenesulfonic acid
δ Chemical shift

### PREPARATION 1

### (2-Bromo-4-methylphenyl)methanol

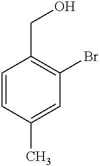

Triethylamine (5.45 mL, 39 mmol) was added to a suspension of 2-bromo-4-methylbenzoic acid (8.0 g, 37.2 mmol) in toluene (200 mL), and the mixture stirred for 5 minutes. Ethyl chloroformate (3.75 mL, 39 mmol) was added and the reaction stirred at room temperature for 90 minutes. Toluene was removed under reduced pressure and the residue re-dissolved in tetrahydrofuran (100 mL). This solution was added dropwise to a solution of lithium aluminium hydride (40 mL, 1M in tetrahydrofuran, 40 mmol) at −78° C., so as to maintain the temperature below −70° C. The reaction was stirred for 30 minutes at this temperature and then allowed to warm to room temperature. The reaction was quenched by the addition of water (1.5 mL), 2N sodium hydroxide solution (1.5 mL) and water (3 mL). The mixture was filtered to remove aluminium salts and the filtrate evaporated under reduced pressure. The residue was purified by column chromatography on silica gel using dichloromethane as eluant to afford the title compound as a solid, 5.03 g, 67%. ¹H NMR (CDCl₃, 400 MHz) δ: 2.32 (s, 3H), 4.70 (s, 2H), 7.12 (d, 1H), 7.33 (d, 1H), 7.37 (s, 1H).

### PREPARATION 2

### tert-Butyl 4-hydroxy-4-[2-(hydroxymethyl)-5-methylphenyl]piperidine-1-carboxylate

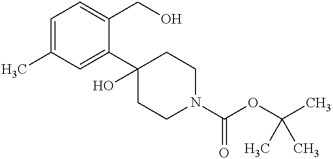

n-Butyl lithium (21 mL, 2.5M in hexane, 52.5 mmol) was added dropwise to a cooled (−65° C.) solution of the alcohol from preparation 1 (5.02 g, 25 mmol) in tetrahydrofuran (25 mL) and ether (25 mL). Once addition was complete, the solution was stirred for 2 hours, allowing to warm to room temperature. The solution was re-cooled to −70° C., and a solution of 1-BOC-4-piperidone (5.47 g, 27 mmol) in tetrahydrofuran (15 mL) and ether (15 mL) was added dropwise, so as to maintain the internal temperature below −65° C. Once addition was complete, the reaction was allowed to Warm to room temperature and stirred for a further 18 hours. The reaction was quenched with 10% citric acid solution and the mixture extracted with ether. The combined organic solutions were washed with brine, dried over Na₂SO₄ and evaporated under reduced pressure. The residual oil was purified by column chromatography using a silica gel cartridge and an elution gradient of dichloromethane:methanol (100:0 to 98:2) to afford the title compound as a clear oil, 3.21 g, 40%. LRMS: m/z ES⁺ 344 [MNa]⁺

### PREPARATION 3

### tert-Butyl 4-hydroxy-4-[3-(hydroxymethyl)pyridin-2-yl]piperidine-1-carboxylate

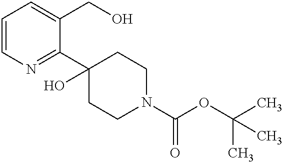

n-Butyl lithium (30 mL, 2.5M in hexane, 75 mmol) was added dropwise to a cooled (−65° C.) solution of (2-bromopyridin-3-yl)methanol (Chem. Pharm. Bull. 38; 9; 1990; 2446) (6.7 g, 35.6 mmol) in tetrahydrofuran (30 mL) and ether (30 mL). Once addition was complete, the solution was stirred for 2 hours. A solution of 1-BOC-4-piperidone (7.8 g, 39.1 mmol) in tetrahydrofuran (10 mL) and ether (10 mL) was added dropwise, so as to maintain the internal temperature below −65° C. Once addition was complete, the reaction was allowed to warm to room temperature and stirred for a further 18 hours. The reaction was quenched with 10% citric acid solution and the mixture extracted with ether (2×75 mL). The combined organic solutions were dried over Na₂SO₄ and evaporated under reduced pressure. The residual oil was purified by column chromatography using a silica gel cartridge and an elution gradient of dichloromethane:methanol (100:0 to 95:5) to afford the title compound as a white solid, 1.33 g, 25%.

¹H NMR (CDCl₃, 400 MHz) δ: 1.47 (s, 9H), 1.62 (m, 2H), 2.24 (m, 2H), 3.28 (m, 2H), 4.03 (m, 2H), 4.87 (s, 2H), 7.23 (m, 1H), 7.80 (m, 1H), 8.47 (m, 1H).

LRMS: m/z ES⁺309 [MH]⁺

### PREPARATION 4

### tert-Butyl 4-hydroxy-4-[2-(2-hydroxyethyl)phenyl]piperidine-1-carboxylate

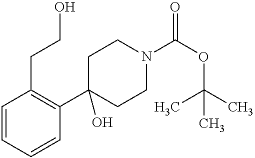

n-Butyl lithium (20.9 mL, 2.5M solution in hexane, 52.25 mmol) was added over 45 minutes to a cooled (−70° C.) solution of 2-bromophenethyl alcohol (5 g, 24.9 mmol) in tetrahydrofuran (25 mL) and ether (25 mL) and the solution then stirred for a further 3 hours. A solution of 4-BOC-1-piperidinone (5.45 g, 27.9 mmol) in tetrahydrofuran (25 mL) was added so as to maintain the temperature below −68° C. and once addition was complete, the reaction was allowed to warm to room temperature, and stirred for a further 18 hours. The reaction was washed with citric acid solution (50 mL), then sodium bicarbonate solution, dried over MgSO₄ and evaporated under reduced pressure. The residual oil was purified by column chromatography on silica gel using dichloromethane:methanol (100:0 to 90:10) as eluant to afford the title compound, 5.9 g, 73.7%. ¹H NMR (CDCl₃, 400 MHz) δ: 1.48 (s, 9H), 1.80-1.90 (m, 2H), 1.95-2.03 (m, 2H), 3.24-3.38 (m, 4H), 3.81 (s, 1H), 3.96-4.10 (m, 4H), 7.19-7.28 (m, 3H), 7.35 (m, 1H). LRMS: m/z APCI⁺ 320 [MH]⁺

### PREPARATION 5

### tert-Butyl 6-methyl-1′H,3H-spiro[2-benzofuran-1,4′-piperidine]-1′-carboxylate

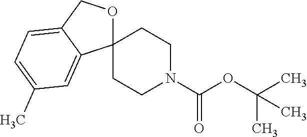

Methane sulphonyl chloride (850 μL, 10.9 mmol) was added to an ice-cold solution of the compound from preparation 2 (3.2 , 9.9 mmol) and triethylamine (2.91 mL, 20.9 mmol) in dichloromethane (25 mL), and the reaction allowed to warm to room temperature and stirred for 18 hours. The reaction was washed with water (10 mL), citric acid solution (10 mL), saturated sodium bicarbonate solution (10 mL) and brine (10 mL), dried over Na₂SO₄ and evaporated under reduced pressure. The residual oil was purified by column chromatography using a silica gel cartridge and an elution gradient of dichloromethane:methanol (100:0 to 98:2) to afford the title compound as a solid, 1.79 g, 59%.

¹H NMR (CDCl₃, 400 MHz) δ: 1.48 (s, 9H), 1.66-1.72 (m, 2H), 1.80 (m, 2H), 2.36 (s, 3H), 3.16 (m, 2H), 4.07 (d, 2H), 5.02 (s, 2H), 6.88 (s, 1H), 7.08 (s, 2H).

LRMS: m/z ES⁺ 326 [MNa]⁺

### PREPARATION 6

### tert-Butyl 1′H,5H-spiro[furo[3,4-b]pyridine-7,4′-piperidine]-1′-carboxylate

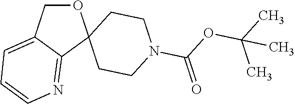

Methane sulphonyl chloride (714 μL, 9.2 mmol) was added to an ice-cold solution of the compound from preparation 3 (2.58 g, 8.4 mmol) and triethylamine (2.45 mL, 17.6 mmol) in dichloromethane (20 mL), and the reaction allowed to warm to room temperature and stirred for 18 hours. Additional methane sulphonyl chloride (357 μL, 4.6 mmol) was added and the reaction stirred for a further 5 hours. The reaction was quenched by the addition of water, and the layers separated. The organic phase was washed with citric acid (10% aq), saturated sodium bicarbonate solution, then brine, dried over Na₂SO₄ and evaporated under reduced pressure. The residual green oil was purified by column chromatography using a silica gel cartridge and an elution gradient of dichloromethane:methanol (100:0 to 96:4) to afford the title compound as a golden oil, 1.05 g. ¹H NMR (CDCl₃, 400 MHz) δ: 1.47 (s, 9H), 1.66 (m, 2H), 2.02 (m, 2H), 3.21 (m, 2H), 4.09 (m, 2H), 5.07 (s, 2H), 7.18 (m, 1H), 7.55 (m, 1H), 8.47 (m, 1H). LRMS: m/z ES⁺ 313 [MNa]⁺

### PREPARATION 7

### tert-Butyl 3,4-dihydro-1′H-spiro[isochromene-1,4′-piperidine]-1′-carboxylate

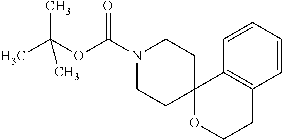

A solution of methane sulphonyl chloride (982 μL, 12.7 mmol) in dichloromethane (20 mL) was added to an ice-cooled solution of the compound from preparation 4 (3.7 g, 11.5 mmol) and triethylamine (3.4 mL, 24.15 mmol) in dichloromethane (20 mL), and the reaction stirred at room temperature for 18 hours. The reaction was washed with water, citric acid solution, sodium bicarbonate solution, then brine, dried over MgSO₄ and evaporated under reduced pressure to provide the title compound as a yellow oil, 3.3 g, 94.5%. ¹H NMR (CDCl₃, 400 MHz) δ: 1.46 (s, 9H), 1.80-1.94 (m, 4H), 2.82 (m, 2H), 3.07-3.22 (m, 2H), 3.86-4.08 (m, 4H), 7.04-7.21 (m, 4H). LRMS: m/z APCI⁺ 304 [MH]⁺

### PREPARATION 8

### 6-Methyl-3H-spiro[2-benzofuran-1,4′-piperidine]

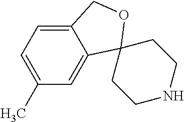

Trifluoroacetic acid (10 mL) was added to a solution of the compound from preparation 5 (1.05 g, 3.6 mmol) in dichloromethane (10 mL) at 0° C. The solution was allowed to warm to room temperature and stirred for 1 hour. The reaction was concentrated under reduced pressure, the residue basified using saturated sodium carbonate solution and the mixture extracted with dichloromethane (2×50 mL). The combined organic extracts were dried over Na₂SO₄ and evaporated under reduced pressure. The residue was purified by column chromatography on silica gel using an elution gradient of dichloromethane:methanol:0.88 ammonia (100:0:0 to 90:10:1) to provide the title compound as a white solid, 785 mg, 65%.

¹H NMR (CDCl₃, 400 MHz) δ: 1.67-1.84 (m, 2H), 1.86-2.03 (m, 2H), 2.36 (s, 3H), 3.05-3.22 (m, 4H), 3.54 (s, 1H), 5.02 (s, 2H), 6.95 (s, 1H), 7.08 (d, 2H).

### PREPARATION 9

### 5H-Spiro[furo[3,4-b]pyridine-7,4′-piperidine]

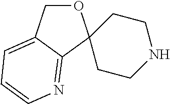

Trifluoroacetic acid (6 mL) was added to a solution of the compound from preparation 6 (1.05 g, 3.6 mmol) in dichloromethane (6 mL) at 0° C. The solution was allowed to warm to room temperature and stirred for 1 hour. The reaction was concentrated under reduced pressure, the residue basified to pH 10 using saturated sodium bicarbonate solution and the mixture extracted with dichloromethane. The combined organic extracts were dried over Na₂SO₄ and evaporated under reduced pressure to afford the title compound as a pale brown gum, 488 mg. ¹H NMR (CDCl₃, 400 MHz) δ: 1.70 (m, 2H), 2.01 (m, 2H), 2.32 (m, 1H), 3.03-3.19 (m, 4H), 5.06 (s, 2H), 7.15 (m, 1H), 7.53 (m, 1H), 8.47 (m, 1H).

LRMS: m/z ES⁺ 191 [MH]⁺

### PREPARATION 10

### 3,4-Dihydrospiro[isochromene-1,4′-piperidine]

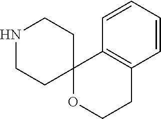

Trifluoroacetic acid (15 mL) was added to a solution of the compound from preparation 7 (3.3 g, 10.87 mmol) in dichloromethane (15 mL) and the reaction stirred at room temperature for 2 hours. The mixture was concentrated under reduced pressure, the residue re-dissolved in dichloromethane and basifled to pH 8 using aqueous sodium carbonate solution. The layers were separated, the organic phase washed consecutively with water, sodium bicarbonate solution and brine, then dried over Na₂SO₄ and evaporated under reduced pressure. The residue was triturated with ether to afford the title compound as a solid, 1.35 g, 64%. ¹H NMR (DMSO-d₆, 400 MHz) δ: 1.88 (m, 2H), 2.09 (m, 2H), 2.77 (t, 2H), 3.02 (m, 2H), 3.15 (m, 2H), 3.90 (t, 2H), 7.10-7.28 (m, 4H).

LRMS: m/z APCI⁺ 204 [MH]⁺

### PREPARATION 11

### 1-Benzyl-4-[(2-bromophenoxy)methyl]-1,2,3,6-tetrahydropyridine

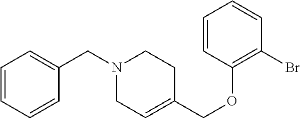

Triphenylphosphine (16.6 g, 63.3 mmol) was added to an ice-cooled solution of 1-benzyl-4-hydroxymethyl-1,2,3,6-tetrahydropyridine (WO 94/20459 page 49) (11.25 g, 55.3 mmol) in tetrahydrofuran (250 mL). 2-Bromophenol (5.57 mL, 52.7 mmol), followed by diisopropyl azodicarboxylate (11.23 mL, 58.0 mmol) were added, the flask wrapped in foil, and the reaction allowed to warm to room temperature, and stirred for a further 18 hours. The foil was removed, the reaction concentrated under reduced pressure and the residue azeotroped with dichloromethane. The residual brown oil was purified by column chromatography using a silica gel cartridge and an elution gradient of pentane:ethyl acetate (90:10 to 50:50) to afford the title compound as a clear oil, 14.92 g, 79%.

¹H NMR (CDCl₃, 400 MHz) δ: 2.28 (m, 2H), 2.63 (m, 2H), 3.03 (m, 2H), 3.60 (s, 2H), 4.47 (s, 2H), 5.82 (m, 1H), 6.81 (m, 1H), 6.88 (m, 1H), 7.20-7.39 (m, 6H), 7.52 (m, 1H). LRMS m/z ES⁺ 358, 360 [MH]⁺

### PREPARATION 12

### 1-Benzyl-4-[(2-bromophenoxy)ethyl]-1,2,3,6-tetrahydropyridine

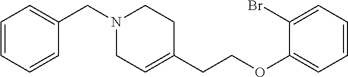

The title compound was prepared as a clear oil in 33% yield from 1,2,3,6-tetrahydro-1-(phenylmethyl)-4-pyridineethanol (WO 01/87838, pg 62) and 2-bromophenol, following the procedure described in preparation 11.

¹H NMR (CDCl₃, 400 MHz) δ: 2.22 (m, 2H), 2.52 (m, 2H), 2.58 (m, 2H), 2.99 (m, 2H), 3.58 (m, 2H), 4.09 (t, 2H), 5.52 (t, 1H), 6.81 (m, 1H), 6.86 (m, 1H), 7.18-7.39 (m, 6H), 7.52 (m, 1H). LRMS: m/z ES⁺ 372, 374 [MH]⁺

### PREPARATION 13

### 1′-Benzylspiro[1-benzofuran-3,4′-piperidine]

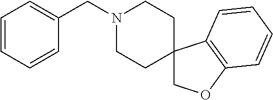

Tributyl tin hydride (22.4 mL, 83.8 mmol) was added to a solution of the compound from preparation 11 (7.46 g, 20.8 mmol) in toluene (1000 mL), and the solution heated under reflux. 2,2′-azobis(2-methylpropionitrile) (690 mg, 4.2 mmol) was added and the reaction heated under reflux for 3 hours. The reaction was cooled to 50° C., concentrated under reduced pressure to a volume of approx 50 mL, this solution diluted with ether (200 mL) and saturated potassium fluoride solution (200 mL) and stirred at room temperature for 18 hours. The layers were separated, the aqueous phase extracted with ether (2×200 mL), the combined organic solutions dried over Na₂SO₄ and concentrated under reduced pressure. The residual oil was purified by column chromatography on a silica gel cartridge using pentane:ethyl acetate (100:0 to 80:20) to afford the title compound as a clear oil, 4.58 g, 78%. ¹H NMR (CDCl₃, 400 MHz) δ: 1.50-1.55 (m, 2H), 1.69-1.76 (m, 2H), 1.94-2.08 (m, 2H), 2.90 (m, 2H), 3.54 (m, 2H), 4.35 (s, 2H), 6.77 (d, 1H), 6.87 (dd, 1H), 7.09-7.39 (m, 7H). LRMS: m/z ES⁺ 280 [MH]⁺

### PREPARATION 14

### 1′-Benzyl-2,3-dihydrospiro[chromene-4,4′-piperidine]

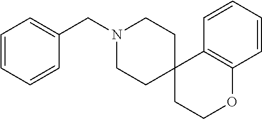

The title compound was obtained as an oil in 56% yield from the compound from preparation 12, following the procedure described in preparation 13. ¹H NMR (CDCl₃, 400 MHz) δ: 1.60 (m, 2H), 1.98 (m, 2H), 2.02-2.33 (m, 4H), 2.43-2.61 (m, 1H), 2.74-2.84 (m, 1H), 3.51-3.63 (m, 2H), 4.11 (m, 2H), 6.78 (m, 1H), 6.85-7.02 (m, 2H), 7.07 (m, 1H), 7.17-7.43 (m, 5H). LRMS: m/z ES⁺ 294 [MH]⁺

### PREPARATION 15

### 1′-Benzyl-6-methoxy-3,4-dihydro-2H-spiro[isoquinoline-1,4′-piperidine]

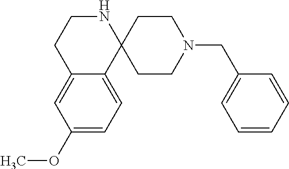

1-Benzyl-4-piperidinone (28.2 g, 149 mmol) was added slowly to a solution of 3-methoxy phenethylamine (23.2 g, 128 mmol) in phosphoric acid (150 mL), and the reaction then heated under reflux for 16 hours. The cooled mixture was poured carefully into ice/water (500 mL), and the mixture diluted with dichloromethane (700 mL). The mixture was basified using concentrated sodium hydroxide solution, with vigorous stirring and then extracted with dichloromethane. The combined organic extracts were evaporated under reduced pressure. The residual oil was purified by column chromatography on silica gel using dichloromethane:methanol (98:2-95:5) as eluant to afford the title compound, 21.4 g, 52%. ¹H NMR (CDCl₃, 400 MHz) δ: 1.65-1.69 (m, 2H), 2.10 (m, 2H), 2.38 (m, 2H), 2.73 (m, 4H), 3.02 (m, 2H), 3.59 (s, 2H), 3.75 (s, 3H), 6.57 (s, 1H), 6.75 (m, 1H), 7.22-7.42 (m, 6H). LRMS: m/z ES⁺ 323.56 [MH]⁺

### PREPARATION 16

### 1′-Benzyl-6-methoxy-2-methyl-3,4-dihydro-2H-spiro[isoquinoline-1,4′-piperidine]

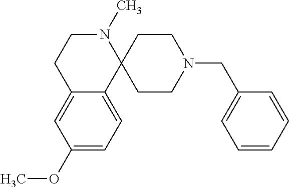

A solution of formaldehyde (49 ml, 33% in water, 0.54 mol) and the amine from preparation 15 (7 g, 21.7 mmol) in methanol (150 mL) was stirred at room temperature for 18 hours. Sodium triacetoxyborohydride (7.1 g, 33.5 mmol) was added and the reaction stirred at room temperature for 4 hours. 10% Sodium carbonate solution was added and the mixture stirred for an hour, and the methanol then evaporated under reduced pressure. The aqueous residue was extracted with dichloromethane, the combined organic extracts washed with water, sodium bicarbonate solution and brine, then dried over MgSO₄ and evaporated under reduced pressure to afford the title compound, as a solid, 5.8 g, 75%. ¹H NMR (CDCl₃, 400 MHz) δ: 1.95-2.10 (m, 4H), 2.30 (s, 3H), 2.57 (m, 2H), 2.74-2.80 (m, 4H), 3.19 (t, 2H), 3.62 (s, 2H), 3.78 (s, 3H), 6.60 (d, 1H), 6.75 (m, 1H), 7.21-7.30 (m, 2H), 7.35 (m, 2H), 7.40 (m, 2H).

LRMS: m/z APCI⁺ 337 [MH]⁺

### PREPARATION 17

### 1′-Benzyl-6-hydroxy-2-methyl-3,4-dihydro-2H-spiro[isoquinoline-1,4′-piperidine]

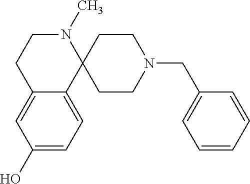

A mixture of the compound from preparation 16 (5.5 g, 16.37 mmol) and hydrobromic acid (30 mL, 48% aq.) in acetic acid (30 mL) was heated under reflux for 22 hours. The cooled mixture was concentrated under reduced pressure and the residue basified to pH 9 using 2M sodium hydroxide solution. The solution was extracted with dichloromethane and the combined organic extracts dried over Na₂SO₄ and evaporated under reduced pressure, to afford the title compound as an oil, 5 g, 95%. ¹H NMR (CDCl₃, 400 MHz) δ: 1.86-2.30 (m, 7H), 2.55-2.82 (m, 6H), 3.15 (t, 2H), 3.69 (s, 2H), 6.50 (s, 1H), 6.62 (m, 1H), 7.04 (m, 1H), 7.21-7.42 (m, 5H). LRMS: m/z APCI⁺ 323 [MH]⁺

### PREPARATION 18

### 1′-Benzyl-2-methyl-3,4-dihydro-2H-spiro[isoquinoline-1,4′-piperidin]-6-yl trifluoromethanesulfonate

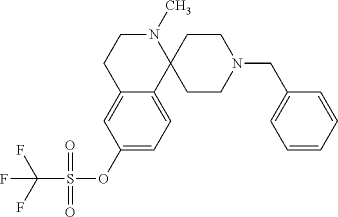

Trifluoromethanesulphonic anhydride (2.9 mL, 17 mmol) was added dropwise to an ice-cooled solution of the compound from preparation 17 (5 g, 15.5 mmol) and N-ethyldiisopropylamine (3.1 mL, 18 mmol) in dichloromethane (50 mL), and once addition was complete, the reaction was stirred for 4 hours at room temperature. The reaction mixture was washed with water (20 mL) and sodium bicarbonate solution (20 mL) then dried over MgSO₄ and evaporated under reduced pressure. The crude product was purified by column chromatography oh silica gel using an elution gradient of dichloromethane:methanol:0.88 ammonia (100:0:0 to 95:5:0.5) to afford the title compound as an oil, 2.9 g, 41%.

¹H NMR (CDCl₃, 400 MHz) δ: 2.10 (m, 2H), 2.23 (s, 3H), 2.60 (m, 2H), 2.82 (m, 2H), 3.16 (m, 2H), 3.18-3.38 (m, 4H), 4.19 (s, 2H), 6.98 (s, 1H), 7.10 (m, 1H), 7.43 (m, 4H), 7.58 (m, 2H). LRMS: m/z APCI⁺ 455 [MH]⁺

### PREPARATION 19

### 1′-Benzyl-2-methyl-3,4-dihydro-2H-spiro[isoquinoline-1,4′-piperidine]

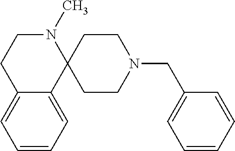

Triethylsilane (1.05 mL, 6.6 mmol) was added to a solution of the compound from preparation 18 (1.2 g, 2.64 mmol), palladium (II) acetate (12 mg, cat.) and 1,3-bis(diphenylphosphino)propane (21.8 mg, cat.) in N,N-dimethylformamide (20 mL) at 60° C., and the reaction stirred for 1 hour. The reaction was concentrated under reduced pressure, the residue re-dissolved in dichloromethane and the organic solution washed with sodium bicarbonate solution and brine. The solution was dried over MgSO₄, and evaporated under reduced pressure. The residual oil was purified by column chromatography on silica gel using an elution gradient of dichloromethane:methanol (100:0 to 90:10) to afford the title compound as an oil, 600 mg, 74%.

LRMS: m/z APCI⁺ 307 [MH]⁺

### PREPARATION 20

### Spiro[1-benzofuran-3,4′-piperidine]

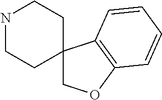

Ammonium formate (1.13 g, 17.94 mmol) was added in one portion to a suspension of the compound from preparation 13 (1.0 g, 3.58 mmol) and 10% palladium on charcoal (750 mg) in ethanol (30 mL), and the reaction heated under reflux for 1.25 hours. The cooled mixture was filtered through Arbocel®, washing through with additional ethanol. The filtrate was evaporated under reduced pressure and the crude product purified by column chromatography on a silica gel cartridge using an elution gradient of dichloromethane:methanol:0.88 ammonia (100:0:0 to 90:10:1) to afford the title compound as a cream coloured solid, 489 mg, 72%. ¹H NMR (CDCl₃, 400 MHz) δ: 1.72 (m, 2H), 1.80-1.96 (m, 2H), 2.70 (m, 2H), 3.11 (m, 2H), 4.40 (s, 2H), 6.79 (m, 1H), 6.88 (m, 1H), 7.13 (m, 1H), 7.14 (m, 1H). LRMS: m/z ES⁺ 190 [MH]⁺

### PREPARATION 21

### 2,3-Dihydrospiro[chromene-4,4′-piperidine]

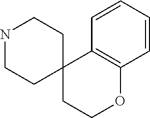

The title compound was obtained as a clear oil in 44% yield from the compound from preparation 14 following the procedure described in preparation 20.

¹H NMR (CDCl₃, 400 MHz) δ: 1.61 (m, 2H), 1.95-2.14 (m, 4H), 2.90 (m, 2H), 3.00 (m, 2H), 4.13 (m, 2H), 6.79 (m, 1H), 6.91 (m, 1H), 7.08 (m, 1H), 7.37 (m, 1H).

LRMS: m/z APCI⁺ 204 [MH]⁺

### PREPARATION 22

### 1H-Spiro[isochromene-4,4′-piperidine]hydrochloride

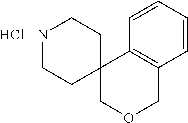

1-Chloroethyl chloroformate (365 mg, 2.55 mmol) was added in one go to an ice-cooled solution of 1′-benzyl-1H-spiro[isochromene-4,4′-piperidine] (Ed. Sci. Farmaco. 1977; 212) (500 mg, 1.7 mmol) and “Proton Sponge” [1,8-bis(dimethylamino)naphthalene] (546 mg, 2.55 mmol) in dichloromethane (5 mL) and the reaction was allowed to warm to room temperature. The reaction was stirred for 45 minutes, then washed with citric acid solution and brine, dried over MgSO₄ and evaporated under reduced pressure. The residue was dissolved in methanol (10 mL) and the solution heated under reflux for 1 hour. The cooled mixture was concentrated under reduced pressure and the residue triturated with ether. The resulting precipitate was filtered off and dried to afford the title compound as a cream coloured solid, 575 mg.

¹H NMR (DMSOd₅, 400 MHz) δ: 1.65-1.80 (m, 2H), 2.15-2.30 (m, 2H), 2.95-3.40 (m, 4H), 3.90 (s, 2H), 4.65 (s, 2H), 6.95-7.05 (d, 1H), 7.10-7.30 (m, 2H), 7.35-7.45 (d, 1H), 8.80-9.20 (m, 2H). LRMS: m/z APCI⁺ 204 [MH]⁺

### PREPARATION 23

### 2-Methyl-3,4-dihydro-2H-spiro[isoquinoline-1,4′-piperidine]dihydrochloride

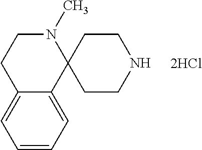

A mixture of the compound from preparation 19 (688 mg, 2.25 mmol) and 10% palladium on charcoal (70 mg) in ethanol:water (6 mL, 90:10) was hydrogenated at 60° C. and 60 psi for 18 hours. The catalyst was filtered off through Arbocel®. Hydrogen chloride in ethanol (1.25M, 3.60 mL, 4.5 mmol) and fresh 10% palladium on charcoal (70 mg) were added to the filtrate and the reaction hydrogenated at 60° C. and 60 psi for a further 18 hours. The reaction was filtered through Arbocel®, the filtrate evaporated under reduced pressure and the residual solid washed with ether and dried in vacuo to afford the title compound as a pale yellow solid, 475 mg, 73%.

LRMS m/z APCI⁺ 217 [MH]⁺

### PREPARATION 24

### N-(6-Methoxypyridin-3-yl)-1′H,3H-spiro[2-benzofuran-1,4′-piperidine]-1′-carbothioamide

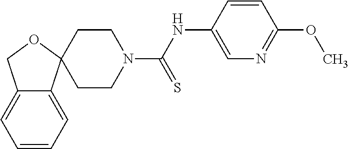

A solution of 5-amino-2-methoxypyridine (278 mg, 2.24 mmol) in dichloromethane (2 mL) was added dropwise to an ice-cold solution of 1,1′-thiocarbonyldi-2(1H)-pyridone (521 mg, 2.24 mmol) in dichloromethane (6 mL) and the resulting orange suspension stirred for 30 minutes. A solution of spiro[isobenzofuran-1(3H), 4′-piperidine (Chem. Pharm. Bull. 46(2); 351-354; 1998) (125 mg, 2.24 mmol) in dichloromethane (2 mL) was added and the reaction allowed to warm to room temperature and stirred for 3 hours. The reaction was washed sequentially with water, sodium carbonate solution, citric acid solution, sodium bicarbonate solution then brine. The solution was dried over MgSO₄ and evaporated under reduced pressure. The residue was triturated with ether, the solid filtered off and dried in vacuo to afford the title compound as a white solid, 379 mg, 47%.

¹H NMR (CDCl₃, 400 MHz) δ: 1.80-1.90 (m, 2H), 2.00-2.10 (m, 2H), 3.55-3.65 (m, 2H), 3.95 (s, 3H), 4.65-4.80 (m, 2H), 5.10 (s, 2H), 6.75-6.80 (d, 1H), 7.05-7.35 (m, 5H), 7.60-7.65 (d, 1H), 8.00 (s, 1H).

LRMS: m/z APCI⁺ 356 [MH]⁺

### PREPARATION 25

### N-(6-Methoxypyridin-3-yl)-3,4-dihydro-1′H-spiro[isochromene-1,4′-piperidine]-1′-carbothioamide

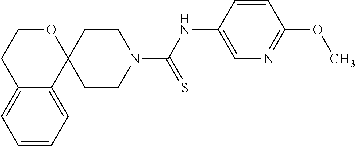

The title compound was obtained as a solid in 56% yield from the compound from preparation 10, following a similar procedure to that described in preparation 24, except the reaction was stirred for 72 hours.

¹H NMR (CDCl₃, 400 MHz) δ: 1.99 (m, 2H), 2.06 (m, 2H), 2.85 (t, 2H), 3.56-3.60 (m, 2H), 3.95 (m, 5H), 4.60 (m, 2H), 6.78 (d, 1H), 7.00 (s, 1H), 7.15 (d, 2H), 7.19 (m, 2H) 7.60 (dd, 1H), 7.99 (d, 1H). LRMS: m/z APCI⁺ 370 [MH]⁺

### PREPARATION 26

### N-(6-Methoxypyridin-3-yl)-1H,1′H-spiro[isochromene-4,4′-piperidine]-1′-carbothioamide

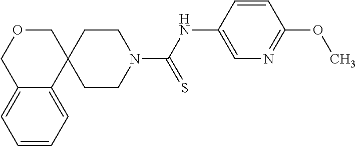

The title compound was obtained as a solid in 93% yield from the compound from preparation 22, following a similar procedure to that described in preparation 24, except N-ethyldiisopropylamine (1.2 eq) was also added to the reaction. ¹H NMR (CDCl₃, 400 MHz) δ: 1.85-2.00 (m, 2H), 2.10-2.25 (m, 2H), 3.40-3.55 (m, 2H), 3.95 (m, 5H), 4.45-4.60 (m, 2H), 4.82 (s, 2H), 6.70-6.80 (d, 1H), 6.95-7.05 (m, 1H), 7.18-7.30 (m, 3H), 7.38-7.45 (d, 1H), 7.60-7.65 (d, 1H), 8.00 (s, 1H). LRMS: m/z APCI⁺ 370 [MH]⁺

### PREPARATION 27

### 6-Fluoro-N-(6-methoxypyridin-3-yl)-1′H,3H-spiro[2-benzofuran-1,4′-piperidine]-1′-carbothioamide

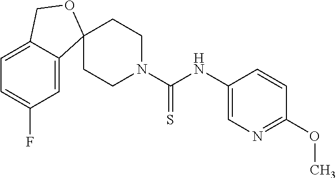

1,1′-Thiocarbonyldi-2(1H)-one (557 mg, 2.4 mmol) was added to a solution of 5-amino-2-methoxypyridine (298 mg, 2.4 mmol) in dichloromethane (15 mL) and the solution stirred for 3 hours. 6-Fluoro-3H-spiro[2-benzofuran-1,4′-piperidine] (WO 04/004714, page 57) (500 mg, 2.4 mmol) was added and the reaction stirred for 18 hours. The reaction was washed with 10% citric acid solution (20 mL), sodium bicarbonate solution (20 mL) and brine (20 mL). The solution was dried over MgSO₄ and evaporated under reduced pressure. The residual foam was purified by column chromatography using a silica gel cartridge and an elution gradient of dichloromethane:methanol (100:0 to 95:5) to afford the title compound as a cream-coloured solid, 624 mg.

¹H NMR (CDCl₃, 400 MHz) δ: 1.85 (m, 2H), 2.00 (m, 2H), 3.58 (m, 2H), 4.02 (s, 3H), 4.81 (m, 2H), 5.06 (s, 2H), 6.81 (d, 1H), 6.85 (d, 1H), 6.98 (m, 1H), 7.18 (m, 1H), 7.15 (m, 1H), 7.98 (d, 1H). LRMS: m/z ES⁺ 396 [MNa]⁺

### PREPARATION 28

### 5-Isothiocyanato-2-methoxypyridine

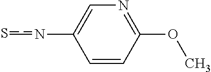

A solution of 5-amino-2-methoxypyridine (5 g, 40.3 mmol) in dichloromethane (25 mL) was added dropwise to an ice-cooled solution of 1,1′-thiocarbonyldi-2(1H)-pyridone (9.36 g, 40.3 mmol) in dichloromethane (25 mL). Once addition was complete, the reaction was allowed to warm to room temperature and stirred for 90 minutes. Additional 5-amino-2-methoxypyridine (2 g, 16 mmol) was added and the reaction stirred for a further 2 hours. The mixture was washed with saturated citric acid solution (25 mL), saturated sodium bicarbonate solution (25 mL) and brine (25 mL). The organic solution was dried over Na₂SO₄ and evaporated under reduced pressure. The residue was dissolved in dichloromethane, and the solution filtered through a pad of silica, washing through with additional dichloromethane. The filtrate was evaporated under reduced pressure to afford the title compound as an off-white solid, 6.13 g, 92%. ¹H NMR (CDCl₃, 400 MHz) δ: 3.92 (s, 3H), 6.72 (d, 1H), 7.41 (dd, 1H), 8.08 (d, 1H). LRMS: m/z ES⁺ 167 [MH]⁺

### PREPARATIONS 29 to 33

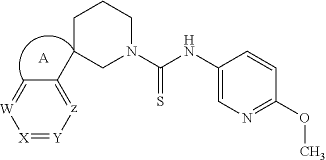

A mixture of the compound from preparation 28 (1 eq) and the appropriate piperidine compounds from preparations 8, 9, 20 and 21 (1 eq) in dichloromethane (3.1 to 6.7 mLmmol⁻¹) was stirred at room temperature for 48 hours. The reaction mixture was evaporated under reduced pressure, the residue triturated with ether and the resulting solid filtered off and dried in vacuo, to afford the title compound as a white solid.

| Prep. No. | 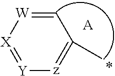 | Data |
|---|---|---|
| 29 | 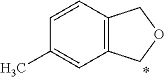 | ¹H NMR (CDCl₃, 400 MHz) δ: 1.82 (m, 2H), 2.02 (m, 2H), 2.37 (s, 3H), 3.57 (m, 2H), 3.93 (s, 3H), 4.67 (m, 2H), 5.06 (s, 2H), 6.75 (m, 1H), 6.93 (s, 1H), 7.02 (bs, 1H), 7.10 (s, 2H), 7.60 (s, 1H), 7.99 (s, 1H). LRMS: m/z APCl⁺ 370 [MH]⁺. 61% yield |
| 30^{A} | 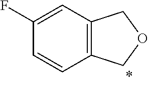 | ¹H NMR (CDCl₃, 400 MHz) δ: 1.82 (m, 2H), 2.00 (m, 2H), 3.55 (m, 2H), 3.93 (s, 3H), 4.68 (m, 2H), 5.06 (s, 2H), 6.75 (m, 1H), 6.91 (m, 1H), 6.95-7.08 (m, 3H), 7.59 (s, 1H), 7.98 (m, 1H). LRMS: m/z APCl⁺ 374 [MH]⁺. 69% yield |
| 31 | 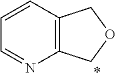 | ¹H NMR (CDCl₃, 400 MHz) δ: 1.82 (m, 2H), 2.23 (m, 2H), 3.68 (m, 2H), 3.94 (s, 3H), 4.69 (m, 2H), 5.11 (s, 2H), 6.76 (m, 1H), 7.18-7.23 (m, 2H), 7.57 (m, 1H), 7.68 (m, 1H), 8.01 (m, 1H), 8.48 (m, 1H). LRMS: m/z APCl⁺ 357 [MH]⁺. 66% yield |
| 32 | 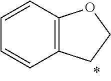 | ¹H NMR (CDCl₃, 400 MHz) δ: 1.75 (m, 2H), 1.88 (m, 2H), 3.28 (m, 2H), 3.83 (s, 3H), 4.50 (s, 2H), 4.74 (m, 2H), 6.78 (m, 2H), 6.86 (m, 1H), 7.13 (m, 1H), 7.28 (m, 1H), 7.63 (m, 1H), 7.97 (m, 1H), 9.27 (s, 1H). LRMS: m/z APCl⁺ 356 [MH]⁺. 94% yield |
| 33 | 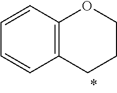 | ¹H NMR (CDCl₃, 400 MHz) δ: 1.66 (m, 2H), 2.00 (m, 2H), 2.09 (m, 2H), 3.31 (m, 2H), 3.84 (s, 3H), 4.14 (m, 2H), 4.71 (m, 2H), 6.73 (m, 1H), 6.77 (m, 1H), 6.88 (m, 1H), 7.07 (m, 1H), 7.34 (m, 1H), 7.64 (m, 1H), 7.98 (m, 1H), 9.24 (s, 1H). LRMS: m/z APCl⁺ 370 [MH]⁺. 88% yield |

^{A}= 5-fluoro-3H-spiro[2-benzofuran-1,4′-piperidine] as described in WO 04/005295, page 69.

### PREPARATION 34

### N-(6-Methoxypyridin-3-yl)-2-methyl-3,4-dihydro-1′H,2H-spiro[isoquinoline-1,4′-piperidine]-1′-carbothioamide

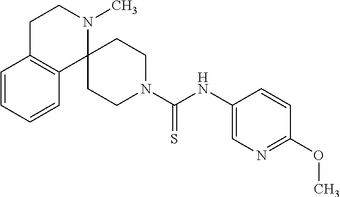

A solution of the isothiocyanate from preparation 28 (266 mg, 1.6 mmol) in dichloromethane (2.5 mL) was added to a solution of the piperidine from preparation 23 (473 mg, 1.6 mmol) and N-ethyldiisopropylamine (692 μL, 4 mmol) in dichloromethane (2.5 mL) and the solution stirred at room temperature for 18 hours. The reaction was diluted with dichloromethane (20 mL), washed sequentially with water (5 mL), sodium bicarbonate solution (5 mL) and brine (5 mL) and dried over MgSO₄. The solution was concentrated under reduced pressure and the crude residue purified by column chromatography on silica gel using dichloromethane:methanol (95:5) as eluant to afford the title compound as a solid, 409 mg, 67%. ¹H NMR (CDCl₃, 400 MHz) δ: 2.08-2.22 (m, 4H), 2.43 (s, 3H), 2.95 (m, 2H), 3.32 (m, 2H), 3.78 (m, 2H), 3.97 (s, 3H), 4.56 (m, 2H), 6.78 (d, 1H), 7.14 (m, 1H), 7.18-7.28 (m, 4H), 7.62 (dd, 1H), 8.00 (d, 1H).

LRMS: m/z APCI⁺ 383 [MH]⁺

### PREPARATIONS 35 to 40

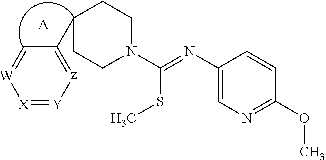

Potassium tert-butoxide (1.2 eq) was added to a solution of the appropriate thioureas from preparations 27 and 29 to 33 (1 eq) in tetrahydrofuran (11.2 to 13.7 mLmmol⁻¹) and the solution stirred for 30 minutes. Methyl-4-toluenesulfonate (1.2 eq) was added and the reaction stirred for between 2 and 3.5 hours, until the reaction was complete. The reaction was diluted with ether, quenched with water, and the layers separated. The aqueous phase was extracted with ether, and the combined organic solutions dried over Na₂SO₄ and evaporated under reduced pressure to afford the desired compounds.

| Prep. No. | 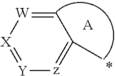 | Data |
|---|---|---|
| 35 | 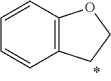 | ¹H NMR (CDCl₃, 400 MHz) δ: 1.79 (m, 2H), 1.97 (m, 2H), 2.12 (s, 3H), 3.42 (m, 2H), 3.91 (s, 3H), 4.33 (m, 2H), 5.05 (s, 2H), 6.68 (m 1H), 6.90 (m, 1H), 6.96 (m, 1H), 7.04-7.14 (m, 2H), 7.76-7.82 (m, 2H). LRMS: m/z APCl⁺ 370 [MH]⁺ |
| 36 | 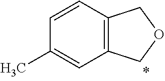 | ¹H NMR (CDCl₃, 400 MHz) δ: 1.78 (m, 2H), 1.96 (m, 2H), 2.12 (s, 3H), 2.37 (s, 3H), 3.41 (m, 2H), 3.91 (s, 3H), 4.32 (m, 2H), 5.05 (s, 2H), 6.68 (dd, 1H), 6.94 (m, 1H), 7.10 (m, 2H), 7.23 (m, 1H), 7.77 (m, 1H). LRMS: m/z APCl⁺ 384 [MH]⁺ |
| 37 | 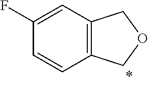 | ¹H NMR (CDCl₃, 400 MHz) δ: 1.78 (m, 2H), 1.94 (m, 2H), 2.11 (s, 3H), 3.39 (m, 2H), 3.91 (s, 3H), 4.32 (m, 2H), 5.05 (s, 2H), 6.67 (dd, 1H), 6.90 (m, 1H), 6.96 (m, 1H), 7.07 (m, 1H), 7.22 (m, 1H), 7.76 (m, 1H). LRMS: m/z APCl⁺ 388 [MH]⁺ |
| 38 | 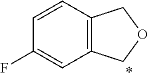 | ¹H NMR (CDCl₃, 400 MHz) δ: 1.78 (m, 2H), 1.91 (m, 2H), 2.09 (s, 3H), 3.38 (m, 2H), 3.89 (s, 3H), 4.33 (m, 2H), 5.02 (s, 2H), 6.65 (d, 1H), 6.82 (dd, 1H), 6.96 (m, 1H), 7.17 (m, 1H), 7.21 (m, 1H), 7.75 (d, 1H). LRMS: m/z APCl⁺ 388 [MH]⁺ |
| 39 | 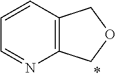 | ¹H NMR (CDCl₃, 400 MHz) δ: 1.76 (m, 2H), 2.11 (s, 3H), 2.17 (m, 2H), 3.41-3.55 (m, 2H), 3.91 (s, 3H), 4.35 (m, 2H), 5.10 (s, 2H), 6.66 (m, 1H), 7.16-7.26 (m, 2H), 7.56 (m, 1H), 7.76 (m, 1H), 8.48 (m, 1H). LRMS: m/z APCl⁺ 371 [MH]⁺ |
| 40 | 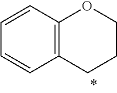 | ¹H NMR (CDCl₃, 400 MHz) δ: 1.71 (m, 2H), 1.84 (m, 2H), 2.09 (m, 2H), 2.14 (s, 3H), 3.22 (m, 2H), 3.91 (s, 3H), 4.17 (m, 2H), 4.27 (m, 2H), 6.68 (m, 1H), 6.81 (m, 1H), 6.92 (m, 1H), 7.10 (m, 1H), 7.15 (m, 1H), 7.73-7.81 (m, 2H). LRMS: m/z APCl⁺ 384 [MH]⁺ |

### PREPARATION 41

### Methyl N-(6-methoxypyridin-3-yl)-2-methyl-3,4-dihydro-1′H,2H-spiro[isoquinoline-1,4′-piperidine]-1′-carbimidothioate

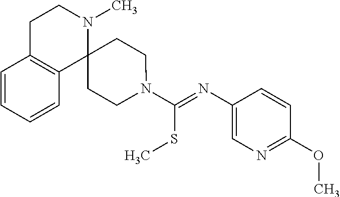

Potassium tert-butoxide (130.6 mg, 1.16 mmol) was added to a solution of the thiourea from preparation 34 (404.6 mg, 1.06 mmol) in tetrahydrofuran (10 mL) and the solution stirred for 30 minutes. Methyl-4-toluenesulfonate (220 mg, 1.16 mmol) was added and the reaction stirred for 3 hours. The reaction was concentrated under reduced pressure and the residue redissolved in dichloromethane (10 mL). The solution was washed with water (3 mL), sodium bicarbonate solution and brine, dried over MgSO₄ and evaporated under reduced pressure to afford the title compound as an oil.

LRMS: m/z ES⁺ 397 [MH]⁺

### EXAMPLES 1 TO 15

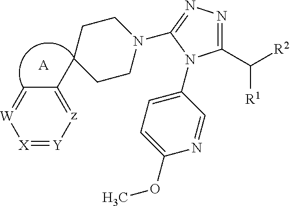

Trifluoroacetic acid (catalytic) was added to a solution of the appropriate compound from preparations 35-41 (1 eq) and commercial acyl hydrazide (R¹R²CHCONHNH₂) (2 eq) in tetrahydrofuran (10-21 mLmmol⁻¹) and the reaction heated at 70° C. for 5 hours, followed by a further 18 hours at room temperature. The reaction was concentrated under reduced pressure, the residue basified using saturated sodium carbonate solution and then extracted with dichloromethane (optionally filtering through a phase separation cartridge). The combined organic extracts were evaporated under reduced pressure and the crude product purified by column chromatography using a silica gel cartridge and an elution gradient of dichloromethane:methanol (100:0 to 95:5) to afford the title compounds.

In the table below, R represents:

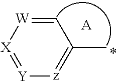

| Ex. No. | Data |
|---|---|
| 1 | R = 2,3-dihydro-1-benzofuran-3-yl; R¹ = H; R² = H. |
| | ¹H NMR (CDCl₃, 400 MHz) δ: 1.68 (m, 2H), 1.86 (m, 2H), |
| | 2.27 (s, 3H), 2.95 (m, 2H), 3.37 (m, 2H), 4.01 (s, 3H), 4.35 (s, |
| | 2H), 6.77 (m, 1H), 6.86 (m, 1H), 6.93 (m, 1H), 7.12 (m, 1H), |
| | 7.62 (m, 1H), 8.17 (m, 1H). LRMS: m/z APCl⁺ 378 [MH]⁺. |
| | 43% yield |
| 2 | R = 2,3-dihydro-1-benzofuran-3-yl; R¹ = OCH₃; R² = H. |
| | ¹H NMR (CDCl₃, 400 MHz) δ: 1.70 (m, 2H), 1.87 (m, 2H), |
| | 2.98 (m, 2H), 3.32 (m, 2H), 3.42 (m, 2H), 4.00 (s, 3H), 4.34 |
| | (s, 2H), 4.25 (s, 2H), 6.78 (s, 1H), 6.86 (s, 1H), 6.90 (m, 1H), |
| | 7.10-7.15 (m, 2H), 7.71 (m, 1H), 8.28 (m, 1H). |
| | LRMS: m/z APCl⁺ 408 [MH]⁺. 42% yield |
| 3^{A} | R = 6-methyl-1,3-dihydro-2-benzofuran-1-yl; R¹ = H; R² = H |
| | ¹H NMR (CDCl₃, 400 MHz) δ: 1.62 (m, 2H), 1.80-1.95 |
| | (m, 2H), 2.24 (s, 3H), 2.34 (s, 3H), 3.27 (m, 4H), 4.00 (s, 3H), |
| | 4.97 (s, 2H), 6.91 (m, 2H), 7.05 (m, 2H), 7.56 (m, 1H), |
| | 8.15 (m, 1H). LRMS: m/z APCl⁺ 392 [MH]⁺. 39% yield |
| 4^{A} | R = 6-methyl-1,3-dihydro-2-benzofuran-1-yl; |
| | R¹ = OCH₃; R² = H |
| | ¹H NMR (CDCl₃, 400 MHz) δ: 1.63 (m, 2H), 1.88 (m, 2H), |
| | 2.34 (s, 3H), 3.26-3.39 (m, 7H), 3.99 (s, 3H), 4.33 (s, 2H), |
| | 4.97 (s, 2H), 6.86-6.95 (m, 2H), 7.06 (m, 2H), 7.68 (m, 1H), |
| | 8.28 (d, 1H). LRMS: m/z APCl⁺ 422 [MH]⁺. 46% yield |
| 5^{A} | R = 5-fluoro-1,3-dihydro-2-benzofuran-1-yl; R¹ = H; R² = H |
| | ¹H NMR (CDCl₃, 400 MHz) δ: 1.62 (m, 2H), 1.85 (m, 2H), |
| | 2.23 (s, 3H), 3.22-3.33 (m, 4H), 3.99 (s, 3H), 4.97 (s, 2H), |
| | 6.91 (m, 1H), 7.02 (m, 2H), 7.55 (m, 1H), 8.14 (m, 1H). |
| | LRMS: m/z APCl⁺ 396 [MH]⁺. 69% yield |
| 6^{A} | R = 5-fluoro-1,3,dihydro-2-benzofuran-1-yl; |
| | R¹ = OCH₃; R² = H |
| | ¹H NMR (CDCl₃, 400 MHz) δ: 1.63 (m, 2H), 1.86 (m, 2H), |
| | 3.24-3.42 (m, 7H), 3.99 (s, 3H), 4.32 (s, 2H), 4.97 (s, 2H), |
| | 6.83-6.96 (m, 3H), 7.03 (m, 1H), 7.66 (m, 1H), 8.27 (m, 1H). |
| | LRMS: m/z APCl⁺ 426 [MH]⁺. 52% yield |
| 7^{B} | R = 5-fluoro-1,3-dihydro-2-benzofuran-1-yl; |
| | R¹ = CH₃; R² = CH₃ |
| | ¹H NMR (CDCl₃, 400 MHz) δ: 1.24 (d, 6H), 1.60 (m, 2H), |
| | 1.76-1.92 (m, 2H), 2.72 (m, 1H), 3.20-3.30 (m, 4H), 4.00 (s, |
| | 3H), 4.97 (s, 2H), 6.82-6.95 (m, 3H), 7.03 (m, 1H), 7.53 |
| | (m, 1H), 8.13 (d, 1H). 23% yield |
| 8 | R = 6-fluoro-1,3-dihydro-2-benzofuran-1-yl; R¹ = H; R² = H. |
| | ¹H NMR (CDCl₃, 400 MHz) δ: 1.62 (m, 2H), 1.82 (m, 2H), |
| | 2.23 (s, 3H), 3.25 (m, 4H), 3.98 (s, 3H), 4.97 (s, 2H), 6.78 |
| | (dd, 1H), 6.93 (m, 2H), 7.58 (dd, 1H), 8.16 (d, 1H). |
| | LRMS: m/z ES⁺ 418 [MNa]⁺. 50% yield |
| 9 | R = 6-fluoro-1,3-dihydro-2-benzofuran-1-yl; |
| | R¹ = OCH₃; R² = H. |
| | ¹H NMR (CDCl₃, 400 MHz) δ: 1.65 (m, 2H), 1.80-1.90 |
| | (m, 2H), 3.25-3.28 (m, 7H), 4.00 (s, 3H), 4.35 (s, 2H), |
| | 4.99 (s, 2H), 6.79 (dd, 1H), 6.88-6.98 (m, 2H), 7.14 (m, 1H), |
| | 7.70 (dd, 1H), 8.26 (d, 1H). LRMS: m/z APCl⁺ 408 [MH]⁺. |
| | 47% yield |
| 10^{A} | R = 5,7-dihydrofuro[3,4-b]pyridin-7-yl; R¹ = H; R² = H |
| | ¹H NMR (CDCl₃, 400 MHz) δ: 1.61 (m, 2H), 2.00 (m, 2H), |
| | 2.22 (s, 3H), 3.24 (m, 2H), 3.30 (m, 2H), 3.97 (s, 3H), 5.01 |
| | (s, 2H), 6.88 (d, 1H), 7.14 (m, 1H), 7.51 (m, 1H), 7.56 (m, 1H), |
| | 8.12 (d, 1H), 8.43 (d, 1H). LRMS: m/z APCl⁺ 379 [MH]⁺. |
| | 38% yield |
| 11^{A} | R = 5,7-dihydrofuro[3,4-b]pyridin-7-yl; R¹ = OCH₃; R² = H |
| | ¹H NMR (CDCl₃, 400 MHz) δ: 1.63 (m, 2H), 2.03 (m, 2H), |
| | 3.22-3.41 (m, 7H), 3.98 (s, 3H), 4.32 (s, 2H), 5.02 (s, 2H), |
| | 6.86 (m, 1H), 7.15 (m, 1H), 7.52 (m, 1H), 7.67 (m, 1H), 8.27 |
| | (m, 1H), 8.44 (m, 1H). LRMS: m/z APCl⁺ 409 [MH]⁺. |
| | 32% yield |
| 12 | R = chroman-4-yl; R¹ = H; R² = H |
| | ¹H NMR (CDCl₃, 400 MHz) δ: 1.55 (m, 2H), 1.94-2.07 |
| | (m, 4H), 2.27 (s, 3H), 3.11 (m, 2H), 3.24 (m, 2H), 4.01 (s, |
| | 3H),4.09 (m, 2H), 6.78 (m, 1H), 6.89 (m, 1H), 6.93 (m, 1H), |
| | 7.07 (m, 1H), 7.25 (m, 1H), 7.63 (m, 1H), 8.17 (m, 1H). |
| | LRMS: m/z APCl⁺ 392 [MH]⁺. 31% yield |
| 13 | R = chroman-4-yl; R¹ = OCH₃; R² = H |
| | ¹H NMR (CDCl₃, 400 MHz) δ: 1.57 (m, 2H), 1.98-2.08 |
| | (m, 4H), 3.15 (m, 2H), 3.28-3.35 (m, 5H), 4.00 (s, 3H), 4.10 |
| | (m, 2H), 4.33 (s, 2H), 6.78 (m, 1H), 6.87-6.92 (m, 2H), |
| | 7.08 (m, 1H), 7.26 (m, 1H), 7.73 (m, 1H), 8.29 (m, 1H). |
| | LRMS: m/z APCl⁺ 422 [MH]⁺. 31% yield |
| 14^{A} | R = 2-methyl-1,2,3,4-tetrahydroisoquinolin-1-yl; |
| | R¹ = H; R² = H |
| | ¹H NMR (CDCl₃, 400 MHz) δ: 1.85-1.98 (m, 4H), 2.24 |
| | (s, 6H), 2.78 (m, 2H), 3.04-3.18 (m, 4H), 3.37-3.44 (m, 2H), |
| | 6.94 (m, 1H), 7.03-7.24 (m, 4H), 7.60 (m, 1H), 8.17 (s, 1H). |
| | LRMS: m/z APCl⁺ 405 [MH]⁺. 22% yield |
| 15^{A} | R = 2-methyl-1,2,3,4-tetrahydroisoquinolin-1-yl; |
| | R¹ = OCH₃; R² = H |
| | ¹H NMR (CDCl₃, 400 MHz) δ: 1.94 (m, 4H), |
| | 2.24 (s, 3H), 2.78 (m, 2H), 3.10-3.19 |
| | (m, 4H), 3.35 (s, 3H), 3.40 (m, 2H), 4.00 (s, |
| | 3H), 4.37 (s, 2H), 6.96 (m, 1H), 7.03-7.23 (m, 4H), 7.69 (m, |
| | 1H), 8.28 (s, 1H). LRMS: m/z APCl⁺ 435 [MH]⁺. 45% yield |

^{A}reaction completed after 5 hours heating
^{B}isobutyric acid hydrazide-see Bioorg. Med. Chem. 11(2003); 1381-87.

### EXAMPLES 16 TO 20

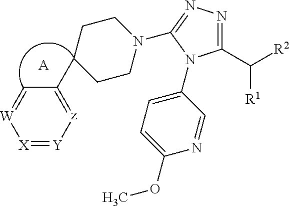

Potassium tert-butoxide (1.05-1.1 eq) was added to an ice-cooled solution of the appropriate thioureas from preparations 24-26 (1 eq) in tetrahydrofuran (4.5-6 mLmmol⁻¹) and the solution allowed to warm to room temperature and stirred for 30 minutes. A solution of methyl-4-toluenesulfonate (1.05-1.1 eq) in tetrahydrofuran (2 mLmmol⁻¹) was added dropwise and the reaction then stirred at room temperature for an hour. The solution was concentrated under reduced pressure and the residue partitioned between ethyl acetate and water. The layers were separated, the organic phase washed with sodium bicarbonate solution and brine, dried over MgSO₄ and evaporated under reduced pressure. The residue was dissolved in tetrahydrofuran (4.5-11.4 mLmmol⁻¹) the appropriate hydrazide (R¹R²CHCONHNH₂) (2 eq) and trifluoroacetic acid (0.5 eq) added and the reaction heated under reflux for up to 18 hours (monitored by tlc to determine when complete). The cooled mixture was evaporated under reduced pressure and the residue partitioned between ethyl acetate and sodium carbonate solution and the layers separated. The organic phase was washed with brine, dried over MgSO₄, and evaporated under reduced pressure. The product was triturated with ether, the solid filtered off and dried in vacuo, to afford the title compound.

In the table below, R represents:

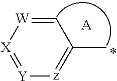

| Ex. No. | Data |
|---|---|
| 16^{A} | R = 1,3-dihydro-2-benzofuran-1-yl; R¹ = H; R² = H. |
| | ¹H NMR (CDCl₃, 400 MHz) δ: 1.60-1.70 (m, 2H), 1.85-2.00 |
| | (m, 2H), 2.25 (s, 3H), 3.30-3.40 (m, 4H), 4.00 (s, 3H), 5.00 (s, |
| | 2H), 6.90-6.95(d, 1H), 7.10-7.30 (m, 4H), 7.60-7.65 (d, 1H), |
| | 8.17 (d, 1H). LRMS: m/z APCl⁺ 378 [MH]⁺. 57% yield |
| 17^{B} | R = 1,3-dihydro-2-benzofuran-1-yl; R¹ = OCH₃; R² = H. |
| | ¹H NMR (CDCl₃, 400 MHz) δ: 1.60-1.70 (m, 2H), 1.85-1.95 |
| | (m, 2H), 3.25-3.40 (m, 7H), 4.00 (s, 3H), 4.35 (s, 2H), |
| | 5.05 (s, 2H), 6.85-6.90 |
| | (d, 1H), 7.10-7.30 (m, 4H), 7.65-7.70 (d, 1H), 8.30 (s, 1H). |
| | LRMS: m/z APCl⁺ 408 [MH]⁺. 68% yield |
| 18 | R = 3,4-dihydro-1H-isochromen-4-yl; R¹ = H; R² = H |
| | ¹H NMR (CDCl₃, 400 MHz) δ: 1.60-1.80 |
| | (m, 2H), 1.90-2.05 (m, |
| | 2H), 2.25 (s, 3H), 3.00-3.15 (m, 2H), 3.18-3.30 (m, 2H), 3.92 |
| | (s, 2H), 4.00 (s, 3H), 4.75 (s, 2H), 6.90-7.00 (m, 2H), 7.10-7.27 |
| | (m, 2H), 7.30-7.40 (d, 1H), 7.50-7.60 (d, 1H), 8.18 (s, 1H). |
| | LRMS: m/z APCl⁺ 392 [MH]⁺. 64% yield |
| 19 | R = 3,4-dihydro-1H-isochromen-4-yl; R¹ = OCH₃; R² = H |
| | ¹H NMR (CDCl₃, 400 MHz) δ: 1.65-1.70 |
| | (m, 2H), 1.90-2.00 (m, |
| | 2H), 3.00-3.15 (m, 2H), 3.20-3.35 (m, 5H), 3.90 (s, 2H), 4.00 (s, |
| | 3H), 4.35 (s, 2H), 4.75 (s, 2H), 6.85-6.95 (m, 2H), 7.10-7.27 |
| | (m, 2H), 7.37 (d, 1H), 7.65-7.70 (d, 1H), 8.28 (s, 1H). |
| | LRMS: m/z APCl⁺ 422 [MH]⁺. 46% yield |
| 20 | R = 3,4-dihydro-1H-isochromen-1-yl; R¹ = H; R² = H |
| | ¹H NMR (CDCl₃, 400 MHz) δ: 1.65-1.70 (m, 2H), 1.90-2.00 |
| | (m, 2H), 2.24 (s, 3H), 2.80 (t, 2H), 3.14-3.19 (m, 2H), 3.28-3.35 |
| | (m, 2H), 3.86 (t, 2H), 4.00 (s, 3H), 6.90 (d, 1H), 7.05-7.20 (m, |
| | 4H), 7.58 (d, 1H), 8.18 (s, 1H). LRMS: m/z APCl⁺ 392 [MH]⁺ |

^{A}= product recrystallised from acetonitrile
^{B}= product purified by column chromatography on silica gel using an elution gradient of ethyl acetate:pentane (50:50 to 100:0)

We claim:

1. A process for the production of alkyl-substituted hydroxyarene compounds comprising the step of treating at least one hydroxyarene with a least one alkylating agent selected from the group consisting of an olefin, alcohol, and ether, in the presence of at least one chloroindate (III) anion containing ionic liquid.

2. The process of claim 1, wherein the hydroxyarene is selected from the group consisting of phenol, cresols, xylenols, trimethylphenols and dihydroxyarenes.

3. The process of claims 1-2 wherein the chloroindate (III) anion is selected from the group consisting of [InCl₄]⁻, [In₂Cl₇]⁻ and [In₃Cl₁₀]⁻.

4. The process of claims 1-3 wherein the alkylating agent is an olefin.

5. The process of claims 1-3 wherein the alkylating agent is an alcohol.

6. The process of claims 1-3 wherein the alkylating agent is an ether.

7. The process of claim 4, wherein the olefin is selected from the group consisting of ethylene, propylene, isobutylene, diisobutylene, cis-2-butene, trans-2-pentene, cyclopentene, cyclohexene, 1,4-cyclohexadiene, and 2-methyl-1-heptene.

8. The process of claim 5, wherein the alcohol is selected from the group consisting of methyl alcohol, ethyl alcohol, n-propyl alcohol, isopropyl alcohol, n-butyl alcohol, sec-butyl alcohol, isobutyl alcohol, t-butyl alcohol, cyclopentyl alcohol, cyclohexyl alcohol and benzyl alcohol.

9. The process of claim 6, wherein the ether is selected from the group consisting of dimethyl ether, ethyl methyl ether, diethyl ether, diisopropyl ether, and methyl t-butyl ether (MTBE).

10. A catalytic ionic liquid system comprising a cationic component and an anionic component, wherein
the cationic component is selected from the group consisting of
(a) an imidazolium cation substituted with 1, 2, or 3-alkyl groups,
(b) tetraalkylphosphonium,
(c) tetraalkylammonium,
(d) dialkylpyrrolidinium, and
(e) piperidinium;
(f) trialkylsulfonium; and
the anionic component is [InₙCl₃ₙ₊₁]⁻, where n is selected from the group consisting of 1, 2, and 3, which is generated by combining InCl₃ and [R] [Cl] where x is the mole fraction of InCl₃ with respect to InCl₃ combined with [R] [Cl], 0.5<x<0.8, and R is selected from either the cationic component or ammonium or phosphonium cations bearing one or more branched alkyl groups, unbranched alkyl groups, cycloalkyl groups, conjugated aryl groups, or unconjugated aryl groups.

11. The catalytic ionic liquid system of claim 10, wherein the catalytic ionic liquid system is formed by the reaction of [R] [Cl] and InCl₃, where [R] is the cationic component.

12. The catalytic ionic liquid system of claim 11 wherein the catalytic ionic liquid system is formed by the reaction of approximately n equivalents of InCl₃ per equivalent of [R] [Cl], where n is 1, 2, or 3.

13. A process for the alkylation of hydroxyarenes comprising a step of treating at least one hydroxyarene with at least one alkylating agent selected from the group consisting of an olefin, alcohol, or ether, in the presence of the catalytic ionic liquid system of claims 10-12.

14. The process of claim 13 comprising a separation step following the step of treating the hydroxyarene with the alkylating agent, wherein the step of treating the hydroxyarene with the alkylating agent yields a product and further wherein:
(i) the separation step uses water;
(ii) the water separates the catalytic ionic liquid system from the product such that the product can be filtered off, and;
(iii) removal of water can regenerate the catalytic ionic liquid system.

15. The process of claim 14, wherein the separation step is decantation.

16. The process of claims 13-15, wherein the reaction of [R][0] and InCl₃ occurs prior to step of treating at least one hydroxyarene with the alkylating agent.

17. The process of claims 13-16 wherein the hydroxyarene is selected from the group consisting of phenol, cresols, xylenols, trimethylphenols and dihydroxyarenes.

18. The process of claims 13-17 wherein the alkylating agent is an olefin selected from the group consisting of ethylene, propylene, isobutylene, diisobutylene, cis-2-butene, trans-2-methyl-1-heptene, cyclohexene.

19. The process of claim 19, wherein the process is carried out at a temperature less than approximately 300° C.

20. The process of claims 13-18, wherein the process is carried out at a temperature of from about 80° C. to about 180° C.

21. The process of claims 13-20, wherein the process is conducted at approximately atmospheric pressure without the use of a high pressure reactor.

22. The process of claims 13-21, wherein the process is carried out at a pressure of approximately 1 atmosphere.

23. The process of claim 13 wherein the hydroxyarene is catechol, the alkylating agent is diisobutylene, and wherein 4-t-octylcatechol is produced.

24. The process of claim 23, wherein the concentration of the catalytic ionic liquid system is from approximately 0.1 mole % to approximately 1 mole % with respect to catechol.

25. The process of claims 23-24 conducted at atmospheric pressure without the use of a high-pressure reactor.

26. The process of claim 13 wherein the hydroxyarene is phenol, the alkylating agent is diisobutylene, and wherein 4-t-octylphenol is produced.

27. The process of claim 26, wherein the concentration of the catalytic ionic liquid system is from approximately 0.1 mole % to approximately 1 mole % with respect to phenol.

28. The process of claim 26-27 conducted at atmospheric pressure without the use of high-pressure reactors.

29. The process of claim 13 wherein the hydroxyarene is m-cresol, the alkylating agent is propylene, and wherein thymol is produced.

30. The process of claim 29, wherein the concentration of the catalytic ionic liquid system is from approximately 0.1 mole % to approximately 1 mole % with respect to m-cresol.

31. The process of claims 29-30 conducted at atmospheric pressure without the use of a high-pressure reactor.

32. The process of claim 1 wherein:
(i) the hydroxyarene is catechol;
(ii) the alkylating agent is t-butanol
(iii) 4-t-butylcatechol is produced; and
(iv) the reaction is carried out at approximately 110° C. and at approximately 1 atmosphere.

33. The process of claim 32, wherein the at least one chloroindate (III) anion containing ionic liquid comprises 1-butyl-3-methylimidazolium heptachlorodiindate (III) or 1,2-dimethyl-3-butylimidazolium heptachlorodiindate (III).

34. The process of claim 1 wherein:
(i) the hydroxyarene is phenol;
(ii) the alkylating agent is t-butanol
(iii) 4-t-butylphenol is produced; and
(iv) the reaction is carried out at approximately 100° C. and at approximately 1 atmosphere.

35. The process of claim 34, wherein the at least one chloroindate (III) anion containing ionic liquid comprises 1-butyl-3-methylimidazolium heptachlorodiindate (III) or 1,2-dimethyl-3-butylimidazolium heptachlorodiindate (III).

36. The process of claim 1 wherein:
(i) the hydroxyarene is p-cresol;
(ii) the alkylating agent is t-butanol
(iii) 2-t-butyl-p-cresol is produced; and
(iv) the reaction is carried out at approximately 100° C. and at approximately 1 atmosphere.

37. The process of claim 36, wherein the at least one chloroindate (III) anion containing ionic liquid comprises 1-butyl-3-methylimidazolium heptachlorodiindate (III) or 1,2-dimethyl-3-butylimidazolium heptachlorodiindate (III).

38. The process of claim 1 wherein:
(i) the hydroxyarene is p-cresol;
(ii) the alkylating agent is t-butanol
(iii) 2,6-di-t-butyl-p-cresol is produced; and
(iv) the reaction is carried out at approximately 100° C. and at approximately 1 atmosphere.

39. The process of claim 38, wherein the at least one chloroindate (III) anion containing ionic liquid comprises 1-butyl-3-methylimidazolium heptachlorodiindate (III) or 1,2-dimethyl-3-butylimidazolium heptachlorodiindate (III).

40. The process of claim 1 wherein:
(i) the hydroxyarene is m-cresol;
(ii) the alkylating agent is isopropanol
(iii) thymol is produced; and
(iv) the reaction is carried out at approximately 100° C. and approximately 1 atmosphere.

41. The process of claim 40, wherein the at least one chloroindate (III) anion containing ionic liquid comprises 1-butyl-3-methylimidazolium heptachlorodiindate (III) or 1,2-dimethyl-3-butylimidazolium heptachlorodiindate (III).

42. The process of claims 1-9, 13-41, further comprising administering microwave energy to at least some of the reactants to affect formation of product.

43. A process for the production of 2-t-butyl-p-cresol comprising the step of treating p-cresol with t-butanol in the presence of at least one chloroindate (III) anion containing ionic liquid, wherein said reaction is carried out at approximately 100° C. and microwave energy is administered to at least some of the reactants to affect formation of product.

44. The process of claim 43, wherein the at least one chloroindate (III) anion containing ionic liquid comprises 1-butyl-3-methylimidazolium heptachlorodiindate (III) or 1,2-dimethyl-3-butylimidazolium heptachlorodiindate (III).
